(19) United States
(12) Patent Application Publication
(10) Pub. No.: US 2020/0188356 A1
(43) Pub. Date: Jun. 18, 2020

(54) Novel Circular RNA Biomarkers for Heart Failure

(71) Applicant: Luxembourg Institute of Health (LIH), Luxembourg (LU)

(72) Inventors: Yvan Devaux, Zoufftgen (FR); Lu Zhang, Metz (FR)

(73) Assignee: Luxembourg Institute of Health (LIH), Luxembourg (LU)

(21) Appl. No.: 16/618,601

(22) PCT Filed: Jun. 1, 2018
(86) PCT No.: PCT/EP2018/064487

§ 371 Date: Dec. 2, 2019

(30) Foreign Application Priority Data
Jun. 2, 2017 (EP) 17174174.7

(51) Int. Cl. A61K 31/403; C12Q 1/6883; A61K 31/40; A61P 9/04

(52) U.S. Cl. CPC A61K 31/403; C12Q 1/6883; C12Q 2600/118; A61P 9/04; C12Q 2600/158; A61K 31/40

(57) ABSTRACT

The application discloses circRNAs as new biomarkers for the diagnosis of heart failure, the prediction of the clinical evolution of heart failure and/or prediction of the response to a treatment in a patient; methods for the prediction of outcome and diagnosis of heart failure and/or predicting the response to a treatment are provided based on measuring said one or more circRNAs; and kits and devices for measuring said circRNAs and/or performing said methods. Further provided are methods for treating patients with heart failure based on the evaluation of said one or more circRNAs.

### FIELD OF THE INVENTION

The invention relates to biomarkers useful for the diagnosis and prediction of diseases and conditions in subjects, in particular heart failure, and to related methods, kits and devices.

### BACKGROUND OF THE INVENTION

Cardiac diseases including stroke continue to be the main cause of death and disability in developed countries. Heart failure (HF), previously called congestive heart failure, is a serious condition most commonly caused by weak pumping of the heart muscle. HF typically results from diseases harming the heart, such as coronary heart disease, especially with a prior heart attack, diabetes, a virus, high blood pressure, cardiomyopathy, arrhythmia, congenital heart defects, heart valve disease or left ventricular reconstruction. For most patients, heart failure is a chronic disease. However, chronic heart failure may decompensate and worsen into acute decompensated heart failure (ADHF), which is a common and potentially fatal cause of acute respiratory distress. Identification of patients at risk of a poor clinical evolution of heart failure or at risk of developing ADHF would be a major step forward towards personalized healthcare as it would allow improving the treatment and follow-up of those patients, thereby stopping the worsening HF and restoring the cardiac and systemic circulation to its chronic, stable state. However, to achieve this goal, novel biomarkers are required.

Since the initial sequencing of the human genome more than a decade ago, huge progress has been made in the understanding of its complexity. It appears now that only a minor part of the human DNA encodes proteins, while the remaining is transcribed into non-protein coding RNAs. Non-coding RNAs have been arbitrarily dichotomized as short non-coding RNAs (20-22 nucleotides-long, called microRNAs, miRNAs) and long non-coding RNAs (IncRNAs, >200 nucleotides).

MicroRNAs (miRNAs) have been the first class of non-coding RNAs reported for their biomarker value and for their ability to predict LV dysfunction after MI. Later on, IncRNAs, either measured in peripheral blood cells or in plasma, have also shown some association with heart failure (reviewed in Devaux Y et al. Nat Rev Cardiol. 2015; 12:415-425).

Circular RNAs (circRNAs) constitute another arm of the family of non-coding RNAs.

Their origin is diverse. They can be produced by the formation of a covalent link between 5′ and 3′ extremities of exons (exonic circRNAs) or introns (intronic circRNAs).

Furthermore, they can be formed by a back-splicing reaction linking exons of protein-coding genes. Exon-intron circRNAs are generated when introns are retained during the circularization of exons. Unlike most IncRNAs, circRNAs are abundant, conserved and stable. In the mammalian brain, circRNAs are dynamically regulated. The function of circRNAs is still poorly characterized, especially in the cardiovascular system. One study has reported an association between a circRNA (a circular form of the IncRNA ANRIL—antisense non-coding RNA in the INK4 locus) and the risk of atherosclerosis (Burd C E, et al. PLoS Genet. 2010; 6:e1001233). An investigation identified a hypoxia-regulated circRNA with proangiogenic activities (Boeckel J N et al. Circ Res 2015; 117:884-90). The heart-related circRNA HRCR acts as a miR-223 sponge and inhibits cardiac hypertrophy and heart failure (Wang K et al. Eur Heart J 2016; 37(33):2602-11).

### SUMMARY OF THE INVENTION

The inventors have identified novel heart failure (HF)-associated circular RNAs (circRNAs). These novel circRNAs are advantageous over previously identified circRNAs, as these novel circRNAs are differentially expressed between subjects with failing hearts and subjects with non-failing hearts and/or are highly expressed in heart tissue. Therefore, these novel circRNAs most likely have a function in the heart and can be used as a biomarker for heart failure. More particularly, these novel circRNAs can be used as biomarkers for the diagnosis of heart failure, for the prediction of the clinical evolution of (chronic) heart failure, such as the prediction of the development of cardiac decompensation i.e. in a patient with chronic heart failure leading to acute heart failure, for selecting an optimal treatment for a patient with heart failure, for use as a valuable therapeutic target in heart failure and/or the prevention of a poor clinical evolution of (chronic) heart failure, in particularly the prevention of the development of cardiac decompensation. For example, the increase or decrease of the expression or activity of these novel circRNAs might be a promising strategy to treat heart failure.

Accordingly, provided herein is the use of one or more circRNAs selected from Table 1, Table 2, Table 3, Table 4, Table 5 and/or Table 6 for diagnosing heart failure and/or predicting the clinical evolution of (chronic) heart failure in a patient and methods based on said use.

A first aspect provides the use of one or more circular RNAs (circRNAs) selected from cFNDC3B, cBPTF, cEXOC6B, cLAMA2-2, cPLCE1 and cPRDM5 for diagnosing heart failure and/or predicting the clinical evolution of heart failure in a patient. In particular embodiments, said use is in vitro or ex vivo. In particular embodiments, the application envisages the one or more circRNAs as described herein, for use in the diagnosis of heart failure and/or for use in predicting the clinical evolution of (chronic) heart failure in a patient.

In particular embodiments, said one or more circRNAs is cFNDC3B and one or more circRNAs selected from cBPTF, cEXOC6B, cLAMA2-2, cPLCE1 and cPRDM5.

In particular embodiments, the use or method further comprises the use of one or more circRNAs selected from Table 1, Table 2, Table 3, or Table 4, in addition to the one or more circRNAs selected from cFNDC3B, cBPTF, cEXOC6B, cLAMA2-2, cPLCE1 and cPRDM5. In particular embodiments, the use or method comprises (i) determining the expression level of said one or more of circRNAs in a sample of said patient with heart failure and optionally (ii) comparing said expression level to the expression level of said one or more circRNAs in a control sample, preferably wherein said diagnosing of heart failure and/or predicting of the clinical evolution of heart failure in said patient is based on the differential expression of said one or more circRNAs. In particular embodiments, said expression level is determined by RT-PCR assay, a sequencing-based assay, a quantitative nuclease-protection assay (qNPA) or a microarray assay. In particular embodiments, the method or use comprises the use comprises determining from said expression levels the differential expression of said one or more circRNAs; said differential expression level diagnosing heart failure and/or predicting the clinical evolution of heart failure in said patient.

In particular embodiments, said use or method further comprises assessing one or more clinical factors in said patient and combining said assessment of said one or more clinical factors and the expression of said one or more circRNAs in said prediction or diagnosis. In particular embodiments, the method is a method of diagnosing heart failure and/or predicting the clinical evolution of (chronic) heart failure in a patient and said clinical factor is selected from the group consisting of breathlessness, exertional dyspnea, orthopnea, paroxysmal nocturnal dyspnea, dyspnea at rest, acute pulmonary edema, chest pain/pressure and palpitations or non-cardiac symptoms such as anorexia, nausea, weight loss, bloating, fatigue, weakness, oliguria, nocturia, cerebral symptoms of varying severity, ranging from anxiety to memory impairment and confusion, fluid retention, cardiac rhythm disturbances, prolonged corrected QT interval and complete Left Bundle Branch Block.

In particular embodiments, the use or method further comprises assessing one or more other biomarkers in said patient and combining said assessment of said one or more other biomarkers and the expression of said one or more circRNAs in said prediction or diagnosis. More particularly, said one or more other biomarkers are selected from the group consisting of long non-coding RNAs, microRNAs, CPK, cTnT, Nt-pro-BNP, MMP9, VEGFB, THBS1 and PIGF.

In particular embodiments, the use or method comprises determining expression of at least 2, 3, 4, 5 or all 6 of cFNDC3B, cBPTF, cEXOC6B, cLAMA2-2, cPLCE1 and cPRDM5; and optionally at least 2, 3, 4, 5, 6, 7, 8, 9, 10 or all 17 of said circRNAs in Table 1, at least 2, 3, 4, 5, 6, 7, 8, 9, 10 or all 765 of said circRNAs in Table 2, at least 2, 3, 4, 5, 6, 7, 8, 9, 10 or all 61 of said circRNAs in Table 3, or at least 2, 3, 4, 5, 6, 7, 8, 9, 10 or all 450 of said circRNAs in Table 4, in addition to said at least 2, 3, 4, 5 or all 6 of cFNDC3B, cBPTF, cEXOC6B, cLAMA2-2, cPLCE1 and cPRDM5. In particular embodiments, said use or method comprises determining the expression of circRNAs in Table 5 and/or Table 6.

In particular embodiments, the methods are carried out on a whole blood sample, preferably a whole blood cell sample.

Also provided herein is a system for diagnosing heart failure and/or predicting clinical evolution heart failure in a patient, the system comprising: a storage memory for storing data associated with a sample obtained from the patient, wherein the data comprises quantitative expression data for one or more circRNAs as taught herein and a processor communicatively coupled to the storage memory for analyzing the dataset, configured to analyse the expression level of said one or more circRNAs and to diagnose heart failure or determine the outcome of heart failure based thereon.

Also provided herein is a computer-readable storage medium storing computer-executable program code, which, when run on a computer allows storing of the data and the analysis of the data in the system according to the methods described herein.

Also provided herein is a kit for diagnosing or predicting the outcome of heart failure in a patient, comprising reagents for specifically determining quantitative expression of one or more circRNAs as taught herein in a sample of a patient and instructions for using said reagents for determining said quantitative expression.

Also provided herein are methods for selecting an optimal treatment for a patient with heart failure, said method comprising determining the risk of a poor clinical evolution of heart failure in said patient using one or more circRNAs as described herein and selecting the treatment for said patient based thereon.

Also provided herein are methods for identifying novel biomarkers for the prognosis and/or diagnosis of heart failure which comprise identifying circRNAs which are (i) differentially expressed in tissue samples from failing and non-failing hearts, (ii) highly expressed and cardiac-enriched, and (iii) expressed in blood samples.

Also provided herein is the use of one or more circRNAs selected from Table 1, Table 2, Table 3, Table 4, Table 5 and/or Table 6, preferably one or more circRNAs selected from cFNDC3B, cBPTF, cEXOC6B, cLAMA2-2, cPLCE1 and cPRDM5,for the treatment of heart failure.

Also provided herein are therapeutic or prophylactic agents for use in the treatment of heart failure, wherein said agent is capable of inhibiting expression or activity of one or more circRNAs selected from Table 1, Table 2, Table 3,Table 4, Table 5 and/or Table 6, preferably one or more circular RNAs (circRNAs) selected from cFNDC3B, cBPTF, cEXOC6B, cLAMA2-2, cPLCE1 and cPRDM5. In particular embodiments, the agent is an agent capable of specifically inhibiting gene expression such as a gene-editing system, an RNAi agent, such as siRNA or shRNA. Also provided are methods for identifying therapeutic or prophylactic agents for use in the treatment of heart failure, wherein said methods comprise determining for a candidate compound whether said compound is capable of inhibiting expression or activity of one or more circRNAs selected from Table 1, Table 2, Table 3, Table 4, Table 5 and/or Table 6, preferably one or more circular RNAs (circRNAs) selected from cFNDC3B, cBPTF, cEXOC6B, cLAMA2-2, cPLCE1 and cPRDM5.

These and further aspects and preferred embodiments are described in the following sections and in the appended claims.

### BRIEF DESCRIPTION OF THE FIGURES

FIG. 1. Pipeline of novel circRNA identification.

FIG. 2. Venn diagrams showing the overlapping circRNAs between different comparisons. (A) 17 circRNAs were obtained from selection 1, (B) 765 circRNAs were obtained from selection 2, (C) 61 circRNAs were obtained from selection 3 and (D) 450 circRNAs were obtained from selection 4. “DE” represents the group of 976 circRNAs, “high” represents the group of 125 circRNAs, “blood” represents the group of 158 circRNAs and “heart” represents the group of 624 circRNAs as identified using the pipeline for identification of novel circRNAs as shown in FIG. 1.

FIG. 3. Table 1: list of 17 novel circRNAs obtained from selection 1.

FIG. 4. Table 2: list of 765 novel circRNAs obtained from selection 2.

FIG. 5. Table 3: list of 61 novel circRNAs obtained from selection 3.

FIG. 6. Table 4: list of 450 novel circRNAs obtained from selection 4.

FIG. 7. Expression profile of circSCNM1 (chr1:151139409-151139890(+)). (A) expression of circSCNM1 in control (ctrl), ICM and DCM as assessed by RNA-seq (expressed in fragment per million reads (FPM)), (B) expression of circSCNM1 in control (ctrl), ICM and DCM as assessed by RNA-seq (expressed in raw reads), (C) expression of circSCNM1in 50 blood samples as assessed by RNA-seq (expressed in raw reads), (D) expression of circSCNM1 in 12 human tissues from public dataset (expressed in raw reads).

FIG. 8. Expression profile of circCHST15 (chr10:125798030-125806240(−)). (A) expression of circCHST15 in control (ctrl), ICM and DCM as assessed by RNA-seq (expressed in fragment per million reads (FPM)), (B) expression of circCHST15 in control (ctrl), ICM and DCM as assessed by RNA-seq (expressed in raw reads), (C) expression of circCHST15in 50 blood samples as assessed by RNA-seq (expressed in raw reads), (D) expression of circCHST15 in 12 human tissues from public dataset (expressed in raw reads).

FIG. 9. Expression profile of circSOX6 (chr11:16205431-16208501(−)). (A) expression of circSOX6 in control (ctrl), ICM and DCM as assessed by RNA-seq (expressed in fragment per million reads (FPM)), (B) expression of circSOX6 in control (ctrl), ICM and

DCM as assessed by RNA-seq (expressed in raw reads), (C) expression of circSOX6 in 50 blood samples as assessed by RNA-seq (expressed in raw reads), (D) expression of circSOX6 in 12 human tissues from public dataset (expressed in raw reads).

FIG. 10. Expression profile of circIFNGR2 (chr21:34804483-34805178(+)). (A) expression of circFNGR2 in control (ctrl), ICM and DCM as assessed by RNA-seq (expressed in fragment per million reads (FPM)), (B) expression of circFNGR2 in control (ctrl), ICM and DCM as assessed by RNA-seq (expressed in raw reads), (C) expression of circFNGR2 in 50 blood samples as assessed by RNA-seq (expressed in raw reads), (D) expression of circFNGR2 in 12 human tissues from public dataset (expressed in raw reads).

FIG. 11. Expression profile of circPHC3 (chr3:169831147-169867032(−)). (A) expression of circPHC3 in control (ctrl), ICM and DCM as assessed by RNA-seq (expressed in fragment per million reads (FPM)), (B) expression of circPHC3 in control (ctrl), ICM and DCM as assessed by RNA-seq (expressed in raw reads), (C) expression of circPHC3 in 50 blood samples as assessed by RNA-seq (expressed in raw reads).

FIG. 12. Expression profile of circPAPD4 (chr5:78952780-78964851(+)). (A) expression of circPAPD4 in control (ctrl), ICM and DCM as assessed by RNA-seq (expressed in fragment per million reads (FPM)), (B) expression of circPAPD4 in control (ctrl), ICM and DCM as assessed by RNA-seq (expressed in raw reads), (C) expression of circPAPD4 in 50 blood samples as assessed by RNA-seq (expressed in raw reads).

FIG. 13. Expression profile of circPCMTD1 (chr8:52773404-52773806(−)). (A) expression of circPCMTD1 in control (ctrl), ICM and DCM as assessed by RNA-seq (expressed in fragment per million reads (FPM)), (B) expression of circPCMTD1 in control (ctrl), ICM and DCM as assessed by RNA-seq (expressed in raw reads), (C) expression of circPCMTD1 in 50 blood samples as assessed by RNA-seq (expressed in raw reads), (D) expression of circPCMTD1 in 12 human tissues from public dataset (expressed in raw reads).

FIG. 14. Expression profile of circAFF2 (chrX:147743428-147744289(+)). (A) expression of circAFF2 in control (ctrl), ICM and DCM as assessed by RNA-seq (expressed in fragment per million reads (FPM)), (B) expression of circAFF2 in control (ctrl), ICM and DCM as assessed by RNA-seq (expressed in raw reads), (C) expression of circAFF2 in 50 blood samples as assessed by RNA-seq (expressed in raw reads), (D) expression of circAFF2 in 12 human tissues from public dataset (expressed in raw reads).

FIG. 15. Expression profile of circCASP1/CARD16 (chr11:104903790-104912446(−)). (A) expression of circCASP1/CARD16 in 50 blood samples as assessed by RNA-seq (expressed in raw reads), (B) expression of circCASP1/CARD16 in 12 human tissues from public dataset (expressed in raw reads).

FIG. 16. Expression profile of circLOC401320 (chr7:30590251-30614497(−)). (A) expression of circLOC401320 in control (ctrl), ICM and DCM as assessed by RNA-seq (expressed in fragment per million reads (FPM)), (B) expression of circLOC401320 in control (ctrl), ICM and DCM as assessed by RNA-seq (expressed in raw reads), (C) expression of circLOC401320 in 50 blood samples as assessed by RNA-seq (expressed in raw reads), (D) expression of circLOC401320 in 12 human tissues from public dataset (expressed in raw reads).

FIG. 17. Expression profile of circFNDC3B (chr3:171965322-171969331(+)). (A) expression of circFNDC3B in control (ctrl), ICM and DCM as assessed by RNA-seq (expressed in fragment per million reads (FPM)), (B) expression of circFNDC3B in control (ctrl), ICM and DCM as assessed by RNA-seq (expressed in raw reads), (C) expression of circFNDC3B in 50 blood samples as assessed by RNA-seq (expressed in raw reads).

FIG. 18. Expression profile of circUBAP2 (chr9:33971648-33973235(−)). (A) expression of circUBAP2 in control (ctrl), ICM and DCM as assessed by RNA-seq (expressed in fragment per million reads (FPM)), (B) expression of circUBAP2 in control (ctrl), ICM and DCM as assessed by RNA-seq (expressed in raw reads), (C) expression of circUBAP2 in 50 blood samples as assessed by RNA-seq (expressed in raw reads).

FIG. 19. Expression profile of circSCMH1 (chr1:41536266-41541123(−)). (A) expression of circSCMH1 in control (ctrl), ICM and DCM as assessed by RNA-seq (expressed in fragment per million reads (FPM)), (B) expression of circSCMH1 in control (ctrl), ICM and DCM as assessed by RNA-seq (expressed in raw reads), (C) expression of circSCMH1 in 50 blood samples as assessed by RNA-seq (expressed in raw reads).

FIG. 20. Expression profile of circRBM23 (chr14:23378691-23380612(−)). (A) expression of circRBM23 in control (ctrl), ICM and DCM as assessed by RNA-seq (expressed in fragment per million reads (FPM)), (B) expression of circRBM23 in control (ctrl), ICM and DCM as assessed by RNA-seq (expressed in raw reads), (C) expression of circRBM23 in 50 blood samples as assessed by RNA-seq (expressed in raw reads).

FIG. 21. Expression profile of MICRA (chr15:64791491-64792365(+)). (A) expression of MICRA in control (ctrl), ICM and DCM as assessed by RNA-seq (expressed in fragment per million reads (FPM)), (B) expression of MICRA in control (ctrl), ICM and DCM as assessed by RNA-seq (expressed in raw reads), (C) expression of MICRA in 50 blood samples as assessed by RNA-seq (expressed in raw reads).

FIG. 22. Expression profile of circBPTF (chr17:65941524-65972074(+)). (A) expression of circBPTF in control (ctrl), ICM and DCM as assessed by RNA-seq (expressed in fragment per million reads (FPM)), (B) expression of circBPTF in control (ctrl), ICM and DCM as assessed by RNA-seq (expressed in raw reads), (C) expression of circBPTF in 50 blood samples as assessed by RNA-seq (expressed in raw reads).

FIG. 23. Expression profile of circCDYL (chr6:4891946-4892613(+)). (A) expression of circCDYL in control (ctrl), ICM and DCM as assessed by RNA-seq (expressed in fragment per million reads (FPM)), (B) expression of circCDYL in control (ctrl), ICM and DCM as assessed by RNA-seq (expressed in raw reads), (C) expression of circCDYL in 50 blood samples as assessed by RNA-seq (expressed in raw reads).

FIG. 24. RNAse R resistance assay. A relative resistance of 5 was taken as a threshold. circRNAs with a relative resistance above 5 were considered resistant. GAPDH and Sf3a1, which are known linear genes, served as control.

FIG. 25. Confirmation of back-splice junctions of 9 RNAse R-resistant circRNAs by Sanger sequencing.

FIG. 26. Expression of (A) circular BPTF, (B) BPTF host linear gene and (C) the ratio of the expression of circular BPTF and its host linear gene, in 5 control (ctrl), 11 ICM and 10 DCM samples as assessed by RNA-seq. (D) Expression of circular BPTF in control (ctrl), ICM and DCM as assessed by qPCR in 66 LV biopsies (23 controls, 26 DCM and 17 ICM). *p<0.05; # p<0.01.

FIG. 27. Expression of (A) circular EXOC6B, (B) EXOC6B host linear gene and (C) the ratio of the expression of circular EXOC6B and its host linear gene, in 5 control (ctrl), 11 ICM and 10 DCM samples as assessed by RNA-seq. (D) Expression of circular EXOC6B in control (ctrl), ICM and DCM as assessed by qPCR in 66 LV biopsies (23 controls, 26 DCM and 17 ICM). *p<0.05; # p<0.01.

FIG. 28. Expression of (A) circular FNDC3B, (B) FNDC3B host linear gene and (C) the ratio of the expression of circular FNDC3B and its host linear gene, in 5 control (ctrl), 11 ICM and 10 DCM samples as assessed by RNA-seq. (D) Expression of circular FNDC3B in control (ctrl), ICM and DCM as assessed by qPCR in 66 LV biopsies (23 controls, 26 DCM and 17 ICM). *p<0.05; # p<0.01.

FIG. 29. Expression of (A) circular LAMA2-2, (B) LAMA2 host linear gene and (C) the ratio of the expression of circular LAMA2-2 and its host linear gene, in 5 control (ctrl), 11 ICM and 10 DCM samples as assessed by RNA-seq. (D) Expression of circular LAMA2-2 in control (ctrl), ICM and DCM as assessed by qPCR in 66 LV biopsies (23 controls, 26 DCM and 17 ICM). *p<0.05; # p<0.01.

FIG. 30. Expression of (A) circular PLCE1, (B) PLCE1 host linear gene and (C) the ratio of the expression of circular PLCE1 and its host linear gene, in 5 control (ctrl), 11 ICM and 10 DCM samples as assessed by RNA-seq. (D) Expression of circular PLCE1 in control (ctrl), ICM and DCM as assessed by qPCR in 66 LV biopsies (23 controls, 26 DCM and 17 ICM). *p<0.05; # p<0.01.

FIG. 31. Expression of (A) circular PRDMS, (B) PRDMS host linear gene and (C) the ratio of the expression of circular PRDMS and its host linear gene, in 5 control (ctrl), 11 ICM and 10 DCM samples as assessed by RNA-seq. (D) Expression of circular PRDMS in control (ctrl), ICM and DCM as assessed by qPCR in 66 LV biopsies (23 controls, 26 DCM and 17 ICM). *p<0.05; # p<0.01.

FIG. 32. Table 5: list of 15 novel circRNAs obtained from the selection of Example 2.

FIG. 33. Table 6 list of 6 of the novel crRNAs obtained from the selection of Example 2.

Abbreviations: ICM, ischaemic cardiomyopathy; DCM, dilated cardiomyopathy; DE, differently expressed; PCMTD1, Protein-L-isoaspartate (D-aspartate) O-methyltransferase domain containing 1; PAPD4, Poly(A) RNA polymerase D4, non-canonical; SOX6, SRY-box 6; IFGR2, Interferon gamma receptor 2; SCNM1, sodium channel modifier 1; PHC3, Polyhomeotic homolog 3; AFF2, AF4/FMR2 family member 2; CHST15, carbohydrate sulfotransferase 15; FNDC3B, Fibronectin type III domain containing 3B; UBAP2, Ubiquitin associated protein; SCMH1, Sex comb on midleg homolog 1; RBM23, RNA binding motif protein 23; ZNF609, Zinc finger protein 609; BPTF, Bromodomain PHD finger transcription factor; CDYL, Chromodomain Y-like; CASP1, Caspace 1; CARD16, caspace recruitment domain family member 16; BPTF, bromodomain PHD finger transcription factor; EXOC6B, exocyst complex component 6B; LAMA2, laminin subunit alpha 2; PLCE1, phospholipase C epsilon 1; PRDMS, PR/SET domain 5; GAPDH, glyceraldehyde-3-phosphate dehydrogenase; Sf3a1, splicing factor 3a subunit 1; PDLIMS, PDZ and LIM domain 5; ARHGAPS, Rho GTPase activating protein 5 ; HERC4, HECT and RLD domain containing E3 ubiquitin protein ligase 4; QKI, QKI, KH domain containing RNA binding; TTN, titin; ctrl, control; FPM, fragment per million reads; circ, circular; c, circular.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The term “biomarker” is widespread in the art and may broadly denote a biological molecule and/or a detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition.

Reference herein to “disease(s) and/or condition(s) as taught herein” or a similar reference encompasses any such diseases and conditions as disclosed herein insofar consistent with the context of such a recitation, in particular heart failure. The term “heart failure” or “HF”, a.k.a. “cardiac failure” or “cardiac dysfunction” as used herein refers to a condition in which the heart is no longer able to pump enough blood to the body's organs and other tissues. As a result thereof, the organs and other tissues do not receive enough oxygen and nutrients to function properly. Possible causes of heart failure are ischemic cardiac disease (myocardial infarction, MI), idiopathic dilated cardiomyopathy (DCM), ischaemic cardiomyopathy (ICM) and hypertrophic cardiomyopathy (HCM), coronary heart diseases, diabetes, a virus, high blood pressure, cardiomyopathy, arrhythmia, congenital heart defects or heart valve disease. Chronic heart failure may decompensate (“cardiac decompensation”) as a reaction to an additional strain on the heart muscle caused by, for instance, an intercurrent illness, myocardial infarction, abnormal heart rhythms, uncontrolled hypertension, diet or medication. Cardiac decompensation in a patient with chronic heart failure typically occurs as an acute event (acute heart failure).

The term “cardiac decompensation” as used herein refers to the inability of the heart to maintain adequate physiological function in the presence of disease, more particularly, to maintain adequate blood circulation after a long-standing cardiovascular pathology.

Cardiac decompensation may also refer to acute decompensated heart failure (ADHF) which is a sudden worsening of the signs and symptoms of heart failure and which may cause acute respiratory distress. The condition is caused by severe congestion of multiple organs by fluid that is inadequately circulated by the failing heart.

The term “dilated cardiomyopathy” or “DCM” relates to a condition whereby the ability of the heart's ventricles and atria to contract is affected leading to heart failure, i.e. the heart is unable to pump sufficiently to maintain blood flow to meet the body's needs. The disease typically starts with dysfunction of the left ventricle. One of the potential effects of left ventricular dysfunction is ventricular remodeling, i.e. changes in ventricular thickness and size which occur as a result of the myocardial damage. Ventricular remodeling occurs at the subcellular, cellular, tissue and chamber level of the heart.

Generally it results in a dilatation and thinning of the ventricular wall as a result of ventricular expansion, and a distortion of the shape of the heart may also occur.

Subsequently, dysfunction of the right ventricle and then the atria occurs. As the heart chambers dilate, contraction of the heart muscle and thereby blood flow from the heart is affected.

The term “ischaemic cardiomyopathy” or “ICM” relates to a condition whereby the narrowing of the coronary arteries which supply blood and oxygen to the heart affect the ability of the heart's ventricles and atria to contract thereby leading to heart failure, i.e. the heart is unable to pump sufficiently to maintain blood flow to meet the body's needs.

The term “hypertrophic cardiomyopathy” or “HCM” relates to a condition whereby at least a part of the myocardium is thickened for no obvious reasons thereby leading to a functional impairment of the cardiac muscle.

The term “myocardial infarction” or “MI” as used herein refers to a condition whereby blood flow to a part of the heart stops causing damage to the heart muscle. MI may be associated with ST elevation (i.e. the trace in the ST segment in the electrocardiogram is abnormally high above the baseline) or can occur without ST segment elevation. The effects of myocardial infarction are diverse. Where the MI is limited, only minor symptoms such as chest pain may occur. Where the MI is significant the damage to the heart muscle affects the function of that part of the heart which, apart from its immediate effect on organ function, may also lead to remodeling of the heart in a way that is further detrimental to its function (e.g. ventricular remodeling as described above).

The terms “predicting” or “prediction” or “prognosis” are commonplace and well-understood in medical and clinical practice. It shall be understood that the terms “predicting and/or prognosticating” may be interchanged with “prediction and/or prognosis” of said disease or condition or “making (or determining or establishing) a prediction and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the condition as taught herein in a subject may also particularly mean that the subject has a ‘positive’ prediction of such, i.e., that the subject is at risk of having such (e.g., the risk is significantly increased vis-à-vis a control subject or subject population). The term “prediction of no” condition as taught herein as described herein in a subject may particularly mean that the subject has a ‘negative’ prediction of such, i.e., that the subject's risk of having such a condition is not significantly increased vis-à-vis a control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

A good prognosis of the condition as taught herein may generally encompass anticipation of a satisfactory partial or complete recovery from the conditions back to before the condition was obtained, preferably within an acceptable time period. A good prognosis of such may more commonly encompass anticipation of not further worsening or aggravating the general health of the patient, preferably within a given time period.

A poor prognosis of the diseases or conditions as taught herein may generally encompass anticipation of a limited recovery and/or unsatisfactorily slow recovery, or to substantially no recovery or even further worsening of such and more particularly resulting in death of the diseased subject.

The term “clinical evolution”, “clinical course”, or “disease outcome” as used herein refers to how a certain disease or condition behaves over time. An unfavourable clinical evolution or poor clinical outcome of the condition as taught herein may generally encompass no recovery, worsening or aggravating of the general health and/or the condition and more particularly resulting in death of the diseased subject. In the context of the present invention, an unfavourable clinical evolution of heart failure may lead to decompensation, such that a prediction or prognosis of the clinical evolution of the disease encompasses predicting the risk or the likelihood of suffering from decompensation. A favourable clinical evolution or good clinical outcome of the condition as taught herein may generally encompass not further worsening or aggravating the general health of the patient, preferably within a given time period.

As used herein, the terms “prevent” and “preventing” in the context of the prognosis of heart failure include the prevention of the worsening of the condition, such as the prevention of decompensation. It is not intended that the present disclosure be limited to complete prevention. In some embodiments, the onset is delayed, or the severity is reduced.

As used herein, the terms “treat” and “treating” are not limited to the case where the subject (e.g., patient) is cured and the condition or disease is eradicated. Rather, embodiments, of the present disclosure also contemplate treatment that merely reduces symptoms, and/or delays conditions or disease progression.

The term “subject” or “patient” as used herein typically denotes humans, but may also encompass reference to non-human animals.

The terms “sample” or “biological sample” as used herein include any biological specimen obtained and isolated from a subject. Samples may include, without limitation, organ tissue (i.e. heart tissue, more particular left ventricle tissue), whole blood, plasma, serum, whole blood cells, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. The term “plasma” defines the colourless watery fluid of the blood that contains in itself no cells, but in which the blood cells (erythrocytes, leukocytes, thrombocytes, etc.) are suspended, containing nutrients, sugars, proteins, minerals, enzymes, etc. Preferred samples in the context of the detection methods of the present invention are blood samples.

The term “tissue” as used herein encompasses all types of cells of the human body including cells of organs but also including blood and other body fluids recited above.

The terms “binding,” “binds,” “recognition,” or “recognize” as used herein are meant to include interactions between molecules that may be detected using, for example, a hybridization assay. When hybridization occurs between two single-stranded polynucleotides, these polynucleotides are described as “complementary”.

Complementarity or homology (the degree that one polynucleotide is complementary with another) can be quantified in terms of the proportion of bases in opposing strands that are expected to form hydrogen bonding with each other, according to generally accepted base-pairing rules.

The term “probe” refers to a molecule capable of hybridizing to a single-stranded nucleic acid target. The probes may target, e.g., comprise a sequence that is the reverse complement of, more than 7, 8, 9, 10, 11, 12, 13, 14, 15, 20, or more (optionally continuous) nucleotides of a given target. The probe may be single stranded nucleic acid sequence and may contain mismatches, additions, or deletions provided the probe retains the ability to bind to the target. In particular embodiments the probe is less than 100, more particularly less than 50 or less than 30 nucleotides.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample is commonly presented as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

The inventors have identified novel circRNAs that can be used as biomarkers for heart failure. These novel circRNAs are advantageous over previously identified circRNAs, as these novel circRNAs are differentially expressed between subjects with failing hearts and subjects with non-failing hearts and/or are highly expressed in heart tissue.

Accordingly, these novel circRNAs most likely have a function in the heart and can be used as a valuable biomarker for heart failure. More particularly, these novel circRNAs can serve as biomarkers for diagnosing heart failure, predicting the clinical evolution of heart failure and predicting the response to treatment, and as therapeutic targets of heart failure. Some of these novel circRNAs were detected in tissue samples from the heart and others in blood samples obtained from said subjects. Therefore, for those novel circRNAs which are detectable in the blood, only a non-invasive and convenient blood sample from a patient would be required for making a diagnosis of heart failure in a patient, for determining the clinical evolution of (chronic) heart failure in a patient and/or determining a method of treatment of heart failure.

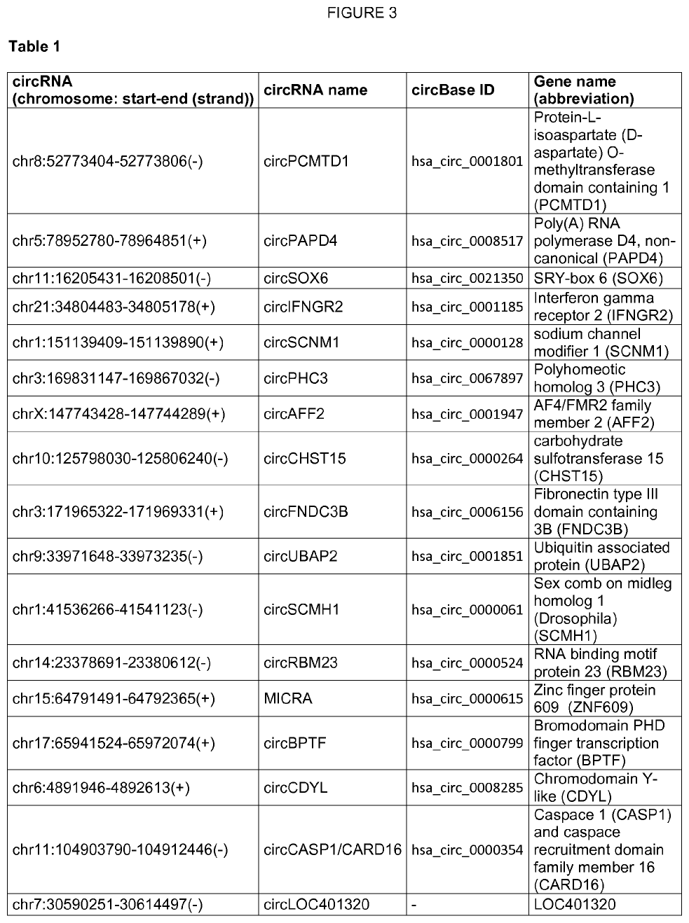

Accordingly, to the first aspect provides the use of one or more circular RNAs (circRNAs) for diagnosing and/or predicting the clinical evolution of heart failure in a patient and methods based on said use or for diagnosing heart failure and/or predicting the clinical evolution of heart failure in a patient. In particular embodiments, the invention envisages methods which are based on determining the expression of one or more circRNAs selected from Table 1, Table 2, Table 3, Table 4, Table 5 and/or Table 6, preferably Table 1, provided in FIGS. 3, 4, 5, 6, 32 and 33, respectively. Accordingly, in particular embodiments, said one or more (such as two, three, four, five, six, seven, eight, nine, ten, twenty, thirty, forty, fifty, sixty, seventy, eighty, ninety, one hundred or more up to all or substantially all) circRNAs are selected from Table 1, Table 2, Table 3, Table 4, Table 5 and/or Table 6. In particular embodiments, said one or more circRNAs are at least 2, 3, 4, 5, 6, 7, 8, 9, 10 or all 17 of said circRNAs in Table 1, at least 2, 3, 4, 5, 6, 7, 8, 9, 10 or all 765 of said circRNAs in Table 2, at least 2, 3, 4, 5, 6, 7, 8, 9, 10 or all 61 of said circRNAs in Table 3, at least 2, 3, 4, 5, 6, 7, 8, 9, 10 or all 450 of said circRNAs in Table 4, at least 2, 3, 4, 5, 6, 7, 8, 9, 10 or all 15 of said circRNAs in Table 5 or at least 2, 3, 4, 5, or all 6 of said circRNAs in Table 6.

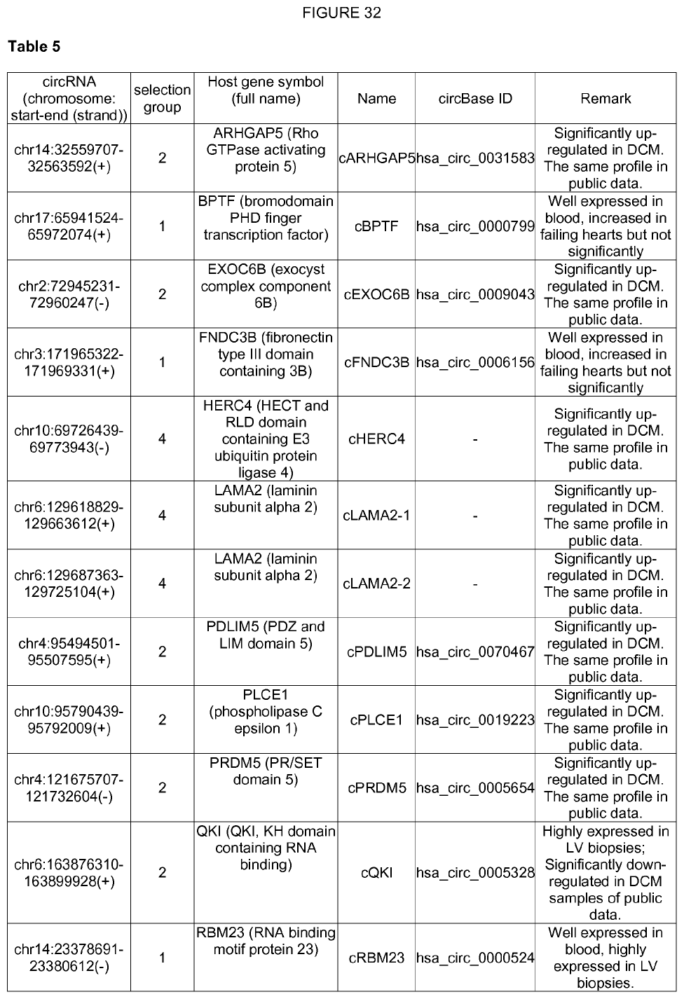

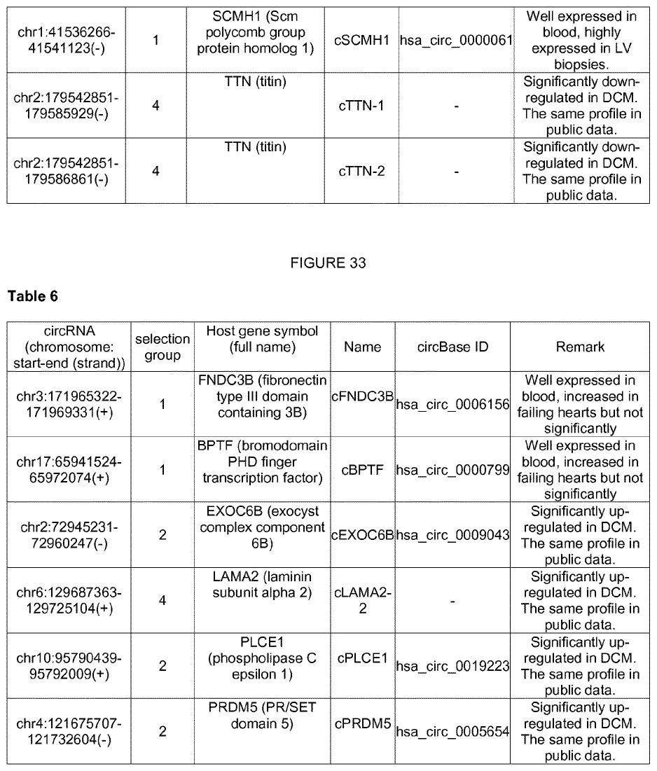

In particular embodiments, said one or more (such as two, three, four, five, six, seven, eight, nine, ten, eleven, twelve, thirteen, fourteen, up to all or substantially all) circRNAs are selected from Table 5, provided in FIG. 32 and/or Table 6 provided in FIG. 33.

Table 5 and 6 provide a further selection of the circRNAs as provided in Tables 1, 2, 3 and 4, as provided in Example 2. This selection includes selecting circRNAs of Tables 1, 2, 3 and 4 which have (i) similar expression profiles between the RNA-seq data of the inventors and public datasets, (ii) high expression level, and (iii) number of circRNAs to be validated kept to a reasonable number.

In particular embodiments, the one or more (such as one, two, three, four, five or all six) circRNAs are selected from cFNDC3B (hsa_circ_0006156), cBPTF (hsa_circ_0000799), cEXOC6B (hsa_circ_0009043), cLAMA2-2, cPLCE1 (hsa_circ_0019223) and cPRDM5 (hsa_circ_0005654), more particularly selected from cFNDC3B, cBPTF, cEXOC6B, cLAMA2-2, cPLCE1 and cPRDM5 as delineated in Table 6, provided in FIG. 33, wherein said cFNDC3B is on chr3:171965322-171969331(+), wherein said cBPTF is on chr17:65941524-65972074(+), wherein said cEXOC6B is on chr2:72945231-72960247(−), wherein said cLAMA2-2 is on chr6:129687363-129725104(+), wherein said cPLCE1 is on chr10:95790439-95792009(+), and wherein said cPRDM5 is on chr4:121675707-121732604(−). The skilled person will understand that “chr” is used to refer to “chromosome”, the first number refers to the start position on said chromosome, the second number refers to the end position on said chromosome and the “(+)” or “(−)” indicates the positive or negative strand of said chromosome, respectively. This also applies to the references to the chromosome start-end and strand indicated for all circRNAs listed in Tables 1, 2, 3, 4, 5 and 6. The codification “hsa_circ_xxxxxxx” (wherein ‘hsa’ refers to homo sapiens and ‘x’ can be any digit) indicates the circBase (http://circbase.org/) identification number annotated under circBase, version of May, 2017 (in which it was indicated that the most recent update of the database took place in December 2015) for a specific human circRNA. If the circRNA was not yet taken up in the circBase, version of May, 2017 (in which it was indicated that the most recent update of the database took place in December 2015), (and is therefore a newly identified circRNA), no circBase identification number is provided herein for said circRNA. This also applies to the references to circBase identification number (“circBase ID”) for all circRNAs listed in Tables 1, 2, 3, 4, 5 and/or 6.

It is noted that a host gene may have more than one circRNA. Therefore, when referring to the circRNAs cFNDC3B, cBPTF, cEXOC6B, cLAMA2-2, cPLCE1 or cPRDM5 or any of the circRNAs as listed in Table 1, Table 2, Table 3, Table 4, Table 5 and/or Table 6, the circRNA identified by the specific chromosomal location (start-end (strand)) and/or the specific circBase identification number indicated in Table 1, Table 2, Table 3, Table 4, Table 5 and/or Table 6, respectively, is intended.

For example, two different circRNAs for host gene LAMA2 are shown in Table 5 (i.e. cLAMA2-1 and cLAMA2-2). When cLAMA2-2 is referred to herein, cLAMA2 identified by chromosomal location chr6:129687363-129725104(+) is intended. On the other hand, only one circRNA for host gene FNDC3B is shown in Table 5 (i.e. cFNDC3B). When cFNDC3B is referred to herein, cFNDC3B identified by chromosomal location chr3:171965322-171969331(+) is intended.

Each of cFNDC3B, cBPTF, cEXOC6B, cLAMA2-2, cPLCE1 and cPRDM5 have reproducible associations with heart failure since they show significantly up-regulated expression levels in both DCM and IDM compared to controls in a cohort of 66 LV biopsies. In addition, cFNDC3B, cBPTF, cEXOC6B, cLAMA2-2, cPLCE1 and cPRDM5 are resistant to RNase R and their back-splice junctions have been confirmed by Sanger sequencing. In particular embodiments, the one or more circRNAs comprise one or more circRNAs selected from cFNDC3B, cBPTF, cEXOC6B, cLAMA2-2, cPLCE1 and cPRDM5, and one or more circRNAs selected from Table 1, Table 2, Table 3 or Table 4, in addition to the one or more circRNAs selected from cBPTF, cEXOC6B, cLAMA2-2, cPLCE1 and cPRDM5.

Of said six circRNAs, cFNDC3B could be of particular interest for the uses and methods as taught herein as cFNDC3B can be detected in a blood sample (which is less invasive for the subject than a biopsy). Furthermore, the FNDC3B gene has long flanking introns, thereby increasing the likelihood of a high ratio of circular FNDC3B over linear FNDC3B (cFNDC3B/FNDC3B).

Accordingly, in particular embodiments, the one or more circRNAs is cFNDC3B and one or more (such as one, two, three, four or all five) circRNAs selected from cBPTF, cEXOC6B, cLAMA2-2, cPLCE1 and cPRDM5; and optionally one or more circRNAs selected from Table 1, Table 2, Table 3 or Table 4, in addition to cFNDC3B and one or more circRNAs selected from cBPTF, cEXOC6B, cLAMA2-2, cPLCE1 and cPRDM5.

Of said six circRNAs, cBPTE could be of particular interest for the uses and methods as taught herein as cBPTE can be detected in a blood sample (which is less invasive for the subject than a biopsy). Furthermore, the cBPTE gene is significantly higher than its linear gene in heart, and the ratio of circular form and its linear gene was also increased in ICM or DCM in RNA-seq data generated from 26 LV biopsies. Accordingly, in particular embodiments, the one or more circRNAs is cBPTF and one or more (such as one, two, three, four or all five) circRNAs selected from cFNDC3B, cEXOC6B, cLAMA2-2, cPLCE1 and cPRDM5; and optionally one or more circRNAs selected from Table 1, Table 2, Table 3 or Table 4, in addition to cBPTF and one or more circRNAs selected from cFNDC3B, cEXOC6B, cLAMA2-2, cPLCE1 and cPRDM5.

Of said six circRNAs, cEXOC6B is of particular interest for the uses and methods as taught herein as cEXOC6B gene is significantly higher than its linear gene in heart.

Accordingly, in particular embodiments, the one or more circRNAs is cEXOC6B and one or more (such as one, two, three, four or all five) circRNAs selected from cBPTF, cEXOC6B, cLAMA2-2, cPLCE1 and cPRDM5; and optionally one or more circRNAs selected from Table 1, Table 2, Table 3 or Table 4, in addition to cEXOC6B and one or more circRNAs selected from cBPTF, cEXOC6B, cLAMA2-2, cPLCE1 and cPRDM5.

Of said six circRNAs, cLAMA2-2 is of particular interest for the uses and methods as taught herein as the ratio of circular form and its linear gene was also increased in ICM or DCM in RNA-seq data generated from 26 LV biopsies. Accordingly, in particular embodiments, the one or more circRNAs is cLAMA2-2 and one or more (such as one, two, three, four or all five) circRNAs selected from cBPTF, cEXOC6B, cLAMA2-2, cPLCE1 and cPRDM5; and optionally one or more circRNAs selected from Table 1, Table 2, Table 3 or Table 4, in addition to cLAMA2-2 and one or more circRNAs selected from cBPTF, cEXOC6B, cLAMA2-2, cPLCE1 and cPRDM5.

Of said six circRNAs, cPLCE1 is of particular interest for the uses and methods as taught herein as cPLCE1 gene has long flanking introns, thereby increasing the likelihood of a high ratio of circular PLCE1 over linear PLCE1 (cPLCE1/PLCE1). Accordingly, in particular embodiments, the one or more circRNAs is cEXOC6B and one or more (such as one, two, three, four or all five) circRNAs selected from cBPTF, cEXOC6B, cLAMA2-2, cPLCE1 and cPRDM5; and optionally one or more circRNAs selected from Table 1, Table 2, Table 3 or Table 4, in addition to cPLCE1 and one or more circRNAs selected from cBPTF, cEXOC6B, cLAMA2-2, cPLCE1 and cPRDM5.

In particular embodiments, the one or more circRNAs is cEXOC6B and one or more (such as one, two, three, four or all five) circRNAs selected from cFNDC3B, cBPTF, cLAMA2-2, cPLCE1 and cPRDM5; and optionally one or more circRNAs selected from Table 1, Table 2, Table 3 or Table 4, in addition to cEXOC6B and one or more circRNAs selected from cFNDC3B, cBPTF, cLAMA2-2, cPLCE1 and cPRDM5.

In particular embodiments, the one or more circRNAs is cLAMA2-2 and one or more(such as one, two, three, four or all five) circRNAs selected from cFNDC3B, cBPTF, cEXOC6B, cPLCE1 and cPRDM5; and optionally one or more circRNAs selected from Table 1, Table 2, Table 3 or Table 4, in addition to cLAMA2-2 and one or more circRNAs selected from cFNDC3B, cBPTF, cEXOC6B, cPLCE1 and cPRDM5.

In particular embodiments, the one or more circRNAs is cPLCE1 and one or more (such as one, two, three, four or all five) circRNAs selected from cFNDC3B, cBPTF, cEXOC6B, cLAMA2-2 and cPRDM5; and optionally one or more circRNAs selected from Table 1, Table 2, Table 3 or Table 4, in addition to cPLCE1 and one or more circRNAs selected from cFNDC3B, cBPTF, cEXOC6B, cLAMA2-2 and cPRDM5.

In particular embodiments, the one or more circRNAs is cPRDM5 and one or more (such as one, two, three, four or all five) circRNAs selected from cFNDC3B, cBPTF, cEXOC6B, cLAMA2-2 and cPLCE1; and optionally one or more circRNAs selected from Table 1, Table 2, Table 3 or Table 4, in addition to cPRDM5 and one or more circRNAs selected from cFNDC3B, cBPTF, cEXOC6B, cLAMA2-2 and cPLCE1.

In particular embodiments, the one or more circRNAs is cFNDC3B and/or cBPTF.

In particular embodiments, the one or more circRNAs are cFNDC3B and cBPTF, and one or more circRNAs selected from cEXOC6B, cLAMA2-2, cPLCE1 and cPRDM5.

Especially the use of at least six specific circRNAs, namely cFNDC3B, cBPTF, cEXOC6B, cLAMA2-2, cPLCE1 and cPRDM5, in generating a differential expression profile (‘biomarker profile’) is envisaged to suffice to successfully diagnose HF and/or predict the clinical evolution of HF in a patient. Accordingly, in particular embodiments, the one or more circRNAs are at least cFNDC3B, cBPTF, cEXOC6B, cLAMA2-2, cPLCE1 and cPRDM5.

Each of these circRNAs is suitable for use in the methods described herein. The methods provided herein involve determining expression or the expression level of one or more circRNAs in a sample. In particular embodiments, the methods comprise detecting the expression or the expression level of one or more biomarkers in a sample or in a tissue of a patient in vitro, ex vivo or in vivo. In particular embodiments, the methods comprise determining the expression or the expression level of a combination of two, three, four, five, six, seven, eight, nine, ten, twenty, thirty, forty, fifty, sixty, seventy, eighty, ninety, one hundred or more up to all or substantially all of the circRNAs of Table 1, Table 2, Table 3, Table 4, Table 5 or Table 6, preferably one or more up to all of the circRNAs of Table 1. In particular embodiments, said circRNAs are one or more circRNAs selected from Table 5 or Table 6, preferably Table 6.

Methods for determining expression of a circRNA are known in the art and include sequencing assays, microarrays, polymerase chain reaction (PCR), RT-PCR, quantitative nuclease-protection assays (qNPA), and Northern blots. Additionally, it can be envisaged that circular RNAs can be detected using, antibody-binding assays, enzyme-linked immunosorbent assays (ELISAs), flow cytometry, protein assays, Western blots, nephelometry, turbidimetry, chromatography, mass spectrometry, or immunoassays. The determination of the expression level of one or more circRNAs can be performed using a singleplex or multiplexed method selected from a group comprising fluorescence, luminescence, radio-marking, next generation sequencing and coded microdisks. Furthermore, the determination of the expression level of said one or more of said circRNAs can also be performed indirectly by measuring abundance levels of cDNAs, amplified RNAs or DNAs or quantities of DNA probes, or other molecules that are indicative of the expression level of the biomarker. The information obtained by the detection method can be quantitative or can be a qualitative signal which can be translated into a quantitative measure by a user or automatically by a reader or computer system. In particular embodiments, the expression of a circRNAs is detected by RT-PCR. In particular embodiments, the circRNAs are detected using probes which specifically detect (and optionally amplify) the junction region of the circRNA.

In the detection methods as envisaged herein the expression of one or more circRNAs is determined in a sample of a subject in vitro or ex vivo or in a tissue in vivo. The subject is preferably a warm-blooded animal, more preferably a mammal, most particularly a human subject, but it can be envisaged that the methods provided herein are equally suitable for methods applied to subjects such as, e.g., non-human primates, equines, canines, felines, ovines, porcines, and the like.

The methods for predicting the clinical evolution and/or outcome of heart failure envisaged herein are particularly suitable when used on a tissue or a sample obtained from a subject that has recently suffered from heart failure. Indeed, in particular embodiments, the methods envisaged herein involve determining the risk of a patient developing heart failure, through for instance left ventricular dysfunction and/or remodelling, after having had a myocardial infarction. In particular embodiments, the patient is a patient who has suffered from a myocardial infarction within less than 5 days, such as less than 3 days, particularly less than 48 hours, such as less than 24 hours before taking of the sample.

Particular embodiments of the invention relate to the use of circRNA for the diagnosis of heart failure. In these embodiments, the patient may be any patient or may be a patient which is characterized by one or more clinical symptoms, such as breathlessness, Exertional dyspnea, Orthopnea, Paroxysmal nocturnal dyspnea, Dyspnea at rest, Acute pulmonary edema, chest pain/pressure and palpitations or noncardiac symptoms such as anorexia, nausea, weight loss, bloating, fatigue, weakness, oliguria, nocturia, fluid retention, cerebral symptoms of varying severity, ranging from anxiety to memory impairment and confusion, cardiac rhythm disturbances, prolonged corrected QT interval and complete Left Bundle Branch Block.

In particular embodiments, the uses and methods as envisaged herein comprise determining the expression or the expression level of one or more circRNAs in a tissue or a sample of a subject and either predicting, based on the result of said determination, the clinical evolution of (chronic) heart failure in a subject, predicting the risk of said subject to develop cardiac decompensation, more particularly, cardiac decompensation as an onset of acute decompensated heart failure (ADHF) or using said information in the diagnosis of heart failure in a subject.

Preferably, said subject, which is to be diagnosed with heart failure, is suspected to have heart failure. More preferably, said subject shows signs and symptoms typical for heart failure.

In particular embodiments, the method may involve comparing the expression level of the one or more circRNAs in a tissue or a sample of a subject with reference values for the expression level of said circRNAs, wherein the reference values represent a known diagnosis of heart failure or a known disease outcome of heart failure, for example, cardiac decompensation or ADHF. In particular embodiments, the uses and methods as taught herein comprise (i) determining the expression level of said one or more of circRNAs in a sample of said patient; and optionally (ii) comparing said expression level to the expression level of said one or more circRNAs in a control sample and (iii) determining from said expression levels the differential expression of said one or more circRNAs; said differential expression level diagnosing heart failure and/or predicting the clinical evolution of heart failure in said patient. For example, distinct reference values may represent the prediction of a risk (e.g., an abnormally elevated risk) of a poor clinical evolution of heart failure, for instance the development of cardiac decompensation vs. the prediction of no or normal risk of a poor clinical evolution of heart failure, for instance the development of cardiac decompensation. In another example, distinct reference values may represent predictions of differing degrees of risk of a poor clinical evolution of heart failure, for instance the development of cardiac decompensation.

Similarly or alternatively, distinct reference values may represent the diagnosis of heart failure vs. the diagnosis of no heart failure (such as, e.g., the diagnosis of healthy, or recovered from heart failure). In another example, distinct reference values may represent the diagnosis of heart failure of varying severity.

In yet another example, distinct reference values may represent a good prognosis for heart failure vs. a poor prognosis for heart failure. In a further example, distinct reference values may represent varyingly favourable or unfavourable prognoses for heart failure. Such comparison may generally include any means to determine the presence or absence of at least one difference and optionally of the size of such difference between values or profiles being compared. A comparison may include a visual inspection, an arithmetical or statistical comparison of measurements. Such statistical comparisons include, but are not limited to, applying an algorithm. If the values or biomarker profiles comprise at least one standard, the comparison to determine a difference in said values or biomarker profiles may also include measurements of these standards, such that measurements of the biomarker are correlated to measurements of the internal standards.

Reference values for the quantity of circRNA expression may be established according to known procedures previously employed for other RNA biomarkers.

For example, a reference value of the amount of circRNA expression for a particular diagnosis, prediction and/or prognosis of heart failure as taught herein may be established by determining the quantity of expression of circRNA in sample(s) from one individual or from a population of individuals characterised by said particular diagnosis, prediction and/or prognosis of said disease or condition. Such population may comprise without limitation 2, 10, 100, or even several hundred individuals or more.

Hence, by means of an illustrative example, reference values of the quantity of circRNA expression for the diagnosis of heart failure vs. no such disease or condition may be established by determining the quantity of circRNA expression in sample(s) from one individual or from a population of individuals diagnosed (e.g., based on other adequately conclusive means, such as, for example, clinical signs and symptoms, imaging, ECG, etc.) as, respectively, having or not having heart failure.

Measuring the expression level of circRNA for the same patient at different time points may in such a case thus enable the continuous monitoring of the status of the patient and may lead to prediction of worsening or improvement of the patient's condition with regard to a given disease or condition as taught herein. Tools such as the kits described herein below can be developed to ensure this type of monitoring. One or more reference values or ranges for circRNA expression levels linked to the presence of heart failure or a poor outcome of heart failure can e.g. be determined beforehand or during the monitoring process over a certain period of time in said subject. Alternatively, these reference values or ranges can be established through data sets of several patients with highly similar disease phenotypes, e.g. from subjects not developing heart failure or from subjects with (chronic) heart failure not developing cardiac decompensation. A sudden deviation of the circRNA levels from said reference value or range can predict the worsening of the condition of the patient (e.g. at home or in the clinic) before the (often severe) symptoms actually can be felt or observed. More particularly, when the presence or absence of heart failure in a subject is evaluated, the reference values or ranges are preferably from subjects not developing heart failure (e.g. healthy subject). On the other hand, when the risk of a poor disease outcome in a subject with (chronic) heart failure, for instance the development of cardiac decompensation, is evaluated, the reference values or ranges are preferably from subjects with (chronic) heart failure with a normal or good disease outcome, for instance, not developing cardiac decompensation.

In particular embodiments, the methods provided herein may include a step of establishing such reference value(s), more particularly a reference value for the expression of one or more circRNAs for the development of heart failure. In particular embodiments, the methods further comprise determining the difference between the quantity of circRNA expression measured in a sample from a subject and the given reference value for said circRNA(s). For example, the difference may represent in the sample of the subject, an increase of at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a the reference value with which a comparison is being made.

Alternatively, such a difference may comprise a decrease in the sample of the subject by, for instance, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a reference value with which a comparison is being made.

The examples section shows that in the experiments done, the increase or decrease in circRNA levels between subjects developing heart failure and subjects not developing heart failure is at least 1.5-fold, i.e. there is at least a 50% increase, or is 0.5-fold or less, i.e. there is at least a 50% decrease, respectively.

In particular embodiments, the preferred circRNAs are those circRNAs which are (i) expressed in blood cells, (ii) differentially expressed between failing and non-failing human hearts or between ICM and DCM, and/or (iii) highly expressed or enriched in the heart. More particularly, the preferred circRNAs are those circRNAs as listed in Table 1.

Even more preferably, the circRNAs are (i) expressed in blood cells, (ii) differentially expressed between failing and non-failing human hearts or between ICM and DCM, and (iii) highly expressed or enriched in the heart.

Preferably, the difference or deviation refers to a statistically significant observed difference. For example, a deviation may refer to an observed difference which falls outside of error margins of reference values in a given population (as expressed, for example, by standard deviation or standard error, or by a predetermined multiple thereof, e.g., ±1xSD or ±2xSD, or ±1xSE or ±2xSE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be established if the observed difference is beyond a given threshold or cut-off. Such threshold or cut-off may be selected as generally known in the art to provide for a chosen sensitivity and/or specificity of the diagnosis, prediction and/or prognosis methods, e.g., sensitivity and/or specificity of at least 50%, or at least 60%, or at least 70%, or at least 80%, or at least 85%, or at least 90%, or at least 95%.

In the methods provided herein the observation of a deviation between the expression of a circRNA in the sample and the reference value (i.e., the differential expression of a circRNA) representing the absence or presence of heart failure in a subject or a good or poor clinical outcome of (chronic) heart failure can lead to the conclusion that the diagnosis or the prediction of the outcome of the condition in said patient is different from that represented by the reference value. Similarly, when no deviation is found between the quantity of expression of a circRNA in a sample from a subject and a reference value representing the absence or presence of heart failure or a good or poor clinical outcome of (chronic) heart failure the absence of such deviation can lead to the conclusion that the diagnosis or the prediction of the outcome the condition in said subject is substantially the same as that represented by the reference value.

The above considerations apply analogously to embodiments wherein different circRNAs are taken into consideration by determining a biomarker profile.

When two or more different biomarkers are determined in a subject, their respective presence, absence and/or quantity may be together represented as a biomarker profile, the values for each measured biomarker making a part of said profile. As used herein, the term “profile” includes any set of data that represents the distinctive features or characteristics associated with a condition of interest, such as with the development of heart failure. Biomarker profiles allow the combination of measurable biomarkers or aspects of biomarkers using methods such as ratios, or other more complex association methods or algorithms (e.g., rule-based methods). A biomarker profile comprises at least two measurements, where the measurements can correspond to the same or different biomarkers. A biomarker profile may also comprise at least three, four, five, 10, 20, 30 or more measurements.

In particular embodiments as described above, the methods envisaged herein comprise determining the expression level of two or more circRNAs for use in a biomarker panel.

Additionally or alternatively other parameters may be used, in combination with the expression of one or more circRNAs as described herein, to diagnose heart failure in a subject or to determine the risk of a poor disease outcome of heart failure in a subject, for example of cardiac decompensation. Taking these additional features into account may further improve the reliability of the assessment. More particularly, where the circRNAs are used in the diagnosis of heart failure, this may be in combination with the assessment of one or more clinical parameters, more particularly clinical parameters which are known in the art to be correlated with heart failure. Examples of such parameters known in the art to be correlated with or indicative of heart failure include but are not limited to breathlessness, Exertional dyspnea, Orthopnea, Paroxysmal nocturnal dyspnea, Dyspnea at rest, Acute pulmonary edema, chest pain/pressure and palpitations or noncardiac symptoms such as anorexia, nausea, weight loss, bloating, fatigue, weakness, oliguria, nocturia, fluid retention, cerebral symptoms of varying severity, ranging from anxiety to memory impairment and confusion, cardiac rhythm disturbances, prolonged corrected QT interval and complete Left Bundle Branch Block. More particularly the parameter may include the observation of the manifestation of one or more of these clinical parameters with progressively increasing severity. Accordingly, in particular embodiments, the invention provides methods for diagnosing heart failure, which methods comprise (i) measuring the expression of one or more circRNAs and (ii) assessing one or more clinical parameters associated with heart failure and determining whether or not the patient is suffering from heart failure based on the outcome of both (i) and (ii).

Where the circRNAs are used for predicting the clinical evolution of (chronic) heart failure, this may be in combination with the assessment of one or more clinical parameters, more particularly clinical parameters which are known in the art to be correlated with a good or poor clinical evolution of (chronic) heart failure. Examples of such clinical parameters include but are not limited to low Left Ventricular Ejection Fraction (LVEF) of ≤40%, anaemia, renal impairment, advanced age, circulating biomarker levels (e.g. brain natriuretic peptides, miRNAs), cardiac rhythm disturbances on the electrocardiogram (ECG), prolonged corrected QT interval (QT_{C}) (e.g. as detectable on ECG), complete Left Bundle Branch Block (LBBB) (e.g. as detectable on ECG) and other parameters obtainable from the interpretation of an ECG. Accordingly, in particular embodiments, the invention provides methods for predicting the clinical evolution of (chronic) heart failure, which methods comprise (i) measuring the expression of one or more circRNAs and (ii) assessing one or more clinical parameters associated with a good and/or poor clinical evolution of (chronic) heart failure and determining whether or not the patient with heart failure is likely to have a good or poor clinical evolution based on the outcome of both (i) and (ii).

Where the circRNAs are used in the prediction of cardiac decompensation, this may be in combination with the assessment of one or more clinical parameters, more particularly clinical parameters which are known in the art to be correlated with cardiac decompensation, more particularly, with acute decompensated heart failure. Examples of such parameters known in the art to be correlated with or indicative of cardiac decompensation include but are not limited to worsening of the parameters correlated with or indicative of heart failure, such as low Left Ventricular Ejection Fraction (LVEF) of ≤40%, anaemia, renal impairment, cardiac rhythm disturbances on the electrocardiogram, prolonged corrected QT interval (QT_{C}), complete Left Bundle Branch Block (LBBB), advanced age and circulating biomarker levels (e.g. brain natriuretic peptides, miRNAs). Accordingly, in particular embodiments, the invention provides methods for predicting cardiac decompensation, which methods comprise (i) measuring the expression of one or more circRNAs and (ii) assessing one or more clinical parameters associated with the development of cardiac decompensation and determining whether or not the patient with heart failure is likely to develop cardiac decompensation based on the outcome of both (i) and (ii).

In particular embodiments, the methods involve taking into account all of these clinical parameters known in the art to be correlated with or indicative of heart failure in combination with one or more circRNAs for the diagnosis of heart failure.

In particular embodiments, the methods involve taking into account all of these clinical parameters known in the art to be correlated with or indicative of a good or poor clinical evolution of (chronic) heart failure in combination with one or more circRNAs for the prediction of the clinical evolution of (chronic) heart failure.

In particular embodiments, the methods involve taking into account all of these clinical parameters known in the art to be correlated with or indicative of cardiac decompensation in combination with one or more circRNAs for the prediction of cardiac decompensation.

Additionally or alternatively other biomarkers may also be used, in combination with the expression of one or more circRNAs as described herein, to diagnose heart failure in a patient or to determine the risk of having a poor clinical evolution of (chronic) heart failure, for instance by worsening of (chronic) heart failure and the development of cardiac decompensation. Any biomarker known to be associated with the occurrence of heart failure or with a poor clinical evolution of (chronic) heart failure, for instance the development of cardiac decompensation, may be suitable in this context. Examples of suitable markers known to be associated with the occurrence of heart failure include but are not limited to long non-coding RNAs, microRNAs such as miR-16, miR-27a, miR-101 and miR-150, all four as described in the European patent application with application number 13802567.1 and the proteins VEGFB, THBS1 and/or PIGF, all three as described in the European patent application with the application number 09752320.3, miR-423, CPK, cTnT, Nt-pro-BNP and MMP9, preferably Nt-pro-BNP. Taking these additional features into account, optionally also in combination with the clinical parameters described above may further improve the reliability of the assessment.

It is envisaged that the methods provided herein which allow the diagnosis of heart failure and/or the identification of patients with heart failure susceptible to a poor clinical outcome can be used to differentiate treatment options for these patients and/or to monitor patients during said treatment. More particularly it is envisaged that identification of patients with heart failure at risk of a poor disease outcome, for instance, at risk of developing cardiac decompensation would allow the treatment of these patients with drugs aimed at countering this poor outcome. Similarly, the diagnosis of patients with heart failure can be used to decide on or confirm the selection of specific therapies aimed at countering heart failure.

Different types of medications have been described which attenuate heart failure and/or a poor clinical evolution of heart failure (e.g. the development of cardiac decompensation), such as but not limited to Angiotensin-converting enzyme (ACE) inhibitors, drugs which directly or indirectly inhibit aldosterone, and certain beta blockers. Indeed, beta-blockers may reverse the remodelling process by reducing left ventricular volumes and improving systolic function. Examples of ACE inhibitors include but are not limited to perindopril, captopril, enalapril, lisinopril, and ramipril. Examples of beta-blockers include but are not limited to carvedilol.

Accordingly, the application also provides methods determining the optimal treatment regimen for a patient with heart failure, more particularly, a patient with heart failure suspected to be at risk of a poor disease outcome, for instance, at risk of developing cardiac decompensation. These methods comprise determining the expression of one or more circRNAs as described hereinabove in a sample of said patient, wherein the selection of treatment is determined based on the expression level of one or more circRNAs so determined. In particular embodiments, the method comprise selecting, where the expression of the one or more circRNA confirms or establishes the diagnosis of heart failure or is indicative of a poor disease outcome for a patient with heart failure, a treatment regimen aimed at countering heart failure, more particularly LVD and/or ventricular remodelling. In further particular embodiments, these methods involve determining whether or not the subject with heart failure is likely to be at risk for a poor disease outcome. In further particular embodiments, these methods include the selection of an anti-remodelling drug for the treatment of those subjects which are determined to be likely to develop heart failure and ventricular remodelling or patients diagnosed with heart failure and/or at risk for a poor disease outcome. Similarly the application provides methods for determining whether or not to treat a patient with a drug which counters heart failure, such as drugs reversing tissue remodelling, such as but not limited to the drugs recited above.

In a related aspect, the application also provides methods determining the efficacy of a treatment regime for a patient with heart failure and/or a patient at risk of a poor clinical evolution of heart failure. These methods comprise determining the expression of one or more circRNAs as described hereinabove in a sample of said patient, wherein the efficacy of treatment is determined based on the expression level of one or more circRNAs so determined.

The present invention further provides systems for diagnosing heart failure in a patient or predicting the clinical evolution of heart failure in a patient, which systems are configured to carry out at least part of the methods described above. Typically, the systems comprise a combination of hardware and software adapted to carry out the determination step described herein.

In particular embodiments, the system comprises a storage memory for storing data associated with a sample obtained from the patient, and a processor communicatively coupled to the storage memory for analyzing the dataset to analyze the expression level of said one or more circRNAs. In particular embodiments, the data comprises quantitative expression data for one or more circRNAs as described herein. In particular embodiments, said circRNAs are selected from Table 1, Table 2, Table 3, Table 4, Table 5 or Table 6, preferably Table 1. In particular embodiments, said circRNAs are selected from Table 5 or Table 6, preferably Table 6.

The system may further comprise hardware means for measuring a signal generated by a sample in a sample container, which signal is indicative of the expression of one or more circRNAs in the sample. In further particular embodiments, the system comprises a detection unit. In particular embodiments, the system further comprises means for separating and optionally identifying the one or more circRNAs from other components present in the sample such as, but not limited to, extraction chambers, chromatography columns, and/or sequencing means.

The application further provides computer-readable storage media storing computer-executable program code, which, when run on a computer allows storing of the data and the analysis of the data in the systems as described above.

The present invention further provides kits or devices for the diagnosis of heart failure, prediction of the clinical evolution of heart failure and/or monitoring of the outcome of heart failure comprising means for detecting the level of one or more circRNAs in a sample of the patient.

In particular embodiments, such a kit or kits of the invention can be used in clinical settings or at home. The kit according to the invention may be used for diagnosing said disease or condition, for monitoring the effectiveness of treatment of a subject suffering from said disease or condition with an agent, or for preventive screening of subjects for the occurrence of said disease or condition in said subject.

Typical kits or devices according to the invention comprise means for measuring the expression of one or more circRNAs in said sample. In particular embodiments, the kits further comprise means for visualizing whether the expression of the one or more circRNAs in said sample is below or above a certain threshold level or value, indicating whether the subject is likely to have heart failure and/or is at risk of a poor outcome of heart failure or not or, where the kit or device is envisaged for diagnosis or prognosis of heart failure, whether the patient is suffering from heart failure or not and/or whether the patient will have a poor clinical evolution of heart failure or not. In particular embodiments, the means may be primers or probes selectively detecting the expression of circRNAs. In further particular embodiments, the probes or primers may be bound on a carrier.

In any of the embodiments of the invention, the kits or devices may additionally comprise one or more selected from means for collecting a sample from the patient, means for communicating directly with a medical practitioner, an emergency department of the hospital or a first aid post, indicating that a person is suffering from said disease or condition or not and/or whether the patient will have a poor clinical evolution of heart failure or not.

The term “threshold level or value” or “reference value” is used interchangeably as a synonym and is as defined herein. It may also be a range of base-line (e.g. “dry weight”) values determined in an individual patient or in a group of patients with highly similar disease conditions.

In any of the embodiments of the invention, the device or kit or kits of the invention can additionally comprise means for detecting the level of an additional marker in the sample of said patient. Non limiting examples of additional markers include but are not limited to long non-coding RNA, microRNA such as miR-423, miR-16, miR-27a, miR-101 and miR-150 and proteins such as CPK, cTnT, Nt-pro-BNP, MMP9, VEGFB, THBS1 and PIGF. In particular embodiments, the kits are envisaged for use in the diagnosis of heart failure or the prognosis of the outcome of heart failure, more particularly to predict the likeliness of a patient to develop cardiac decompensation.

The invention further provides combinations of probes for use in the detection of the expression of one or more circRNAs in a sample of a patient, more particularly for the diagnosis of heart failure in a patient or for determining the likeliness of the patient to have a poor outcome of heart failure. More particularly, these probes can be used to selectively detect the expression of one or more circRNAs. In further particular embodiments, these probes are provided on a substrate. Examples of suitable substrate materials include but are not limited to glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, polymers and multiwell (e.g. microtiter) plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon

The application envisages both in vitro and in vivo methods of detection of the biomarkers described herein. Accordingly, it is evident that the application also relates to the biomarkers described herein for use in the methods of diagnosis or prognosis as described hereinabove.

The application further provides methods for treatment which are based on the upregulation or downregulation of expression of one or more circRNAs described herein.

Indeed, given that circRNAs have been found to have strong regulatory functions in gene expression and as the inventors have identified a number of circRNAs which are significantly correlated with heart failure and/or the clinical evolution thereof, it is envisaged that heart failure and/or the risk of a poor outcome of heart failure can be influenced by the expression of circRNAs. Accordingly, methods of treatment involving regulation of expression of the circRNAs described herein are also envisaged. In particular embodiments, upregulation of expression of circRNAs associated with the absence of heart failure or a good outcome of heart failure is envisaged. In further embodiments, downregulation of expression of circRNAs associated with the presence of heart failure and/or a poor outcome of heart failure is envisaged. Methods for increasing or decreasing expression of circRNAs include gene modulation or modification technologies such as, but not limited to CRISPR-based technologies.

The invention further provides methods for identifying compounds capable of reducing heart failure and/or the risk of a poor outcome of heart failure, which methods are based on detecting circRNA expression. Based on the circRNAs identified herein, it is possible to screen for agents which can decrease heart failure and/or the risk of a poor outcome of heart failure,. The screening methods envisaged herein may involve detecting the expression of one or more of the circRNA identified herein in an animal model for heart failure, in controls and upon treatment with one or more test agents. Test agents capable of inducing an expression profile which is linked to the absence of heart failure and/or a decreased risk of a poor outcome of heart failure can be identified as agents of interest in the treatment of heart failure. The application further relates to the use of one or more circRNAs of Table 1, Table 2, Table 3, Table 4, Table 5 or Table 6, for the treatment of heart failure. In particular embodiments, said one or more circRNAs are selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654).

Furthermore, another aspect of the invention is a therapeutic or prophylactic agent for use in the treatment of heart failure, wherein said agent is capable of inhibiting expression or activity of one or more circRNAs of Table 1, Table 2, Table 3, Table 4, Table 5 or Table 6, preferably Table 1. In particular embodiments, wherein said agent is capable of inhibiting expression or activity of one or more circRNAs of Table 5 or Table 6, preferably Table 6. In particular embodiments, wherein said agent is capable of inhibiting expression or activity of one or more circRNAs selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654).

As used herein, the term “agent” broadly refers to any chemical (e.g., inorganic or organic), biochemical or biological substance, compound, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, peptides, peptidomimetics, antibodies and fragments and derivatives thereof, aptamers, photoaptamers, chemical substances, preferably organic molecules, more preferably small organic molecules, lipids, carbohydrates, polysaccharides, etc., and any combinations thereof.

The term “inhibit” as used herein is intended to be synonymous with terms such as “decrease”, “reduce”, “diminish”, “interfere”, “disrupt”, or “disturb”, and denotes a qualitative or quantitative decrease of the expression and/or activity of one or more circRNAs that is being interfered with. The term encompasses any extent of such interference. For example, the interference may encompass a decrease of at least about 10%, e.g., of at least about 20%, of at least about 30%, e.g., of at least about 40%, of at least about 50%, e.g., of at least about 60%, of at least about 70%, e.g., of at least about 80%, of at least about 90%, e.g., of at least about 95%, such as of at least about 96%, 97%, 98%, 99% or even of 100%, compared to a reference situation without said interference. The skilled person will understand that the methods of measuring the decrease of the expression of circRNAs are the same as for determining the expression of a circRNA as defined elsewhere herein.

The term “promote” as used herein is intended to be synonymous with terms such as “increase”, “elevate”, “boost”, “raise”, or “augment” and denotes a qualitative or quantitative increase of the expression and/or activity of one or more circRNAs that is being promoted. The term encompasses any extent of such promotion. For example, the interference may encompass an increase of at least about 10%, e.g., of at least about 20%, of at least about 30%, e.g., of at least about 40%, of at least about 50%, e.g., of at least about 60%, of at least about 70%, e.g., of at least about 80%, of at least about 90%, e.g., of at least about 100%, of at least about 200%, of at least about 300%, compared to a reference situation without said promotion. The skilled person will understand that the methods of measuring the increase of the expression of circRNAs are the same as for determining the expression of a circRNA as defined elsewhere herein.

In particular embodiments, the therapeutic or prophylactic agent according to the invention is selected from the group consisting of a protein, a polypeptide, a peptide, a peptidomimetic, a nucleic acid, an aptamer, a small organic molecule, and a compound or combination of any two or more thereof; preferably wherein said agent is a gene-editing system, an RNAi agent, such as siRNA or shRNA, or an antibody or functional fragment thereof.

The application further provides methods to identify novel circRNAs which can serve as biomarkers for heart failure, as performed in the example section, can comprise the differential analysis of gene and transcript expression using high-throughput RNA sequencing. Differential analysis can be performed by use of tools developed by the bioinformatics community. For example, the circRNA finder, find_circ, CIRI, MapSplice or CIRCexplorer algorithm.

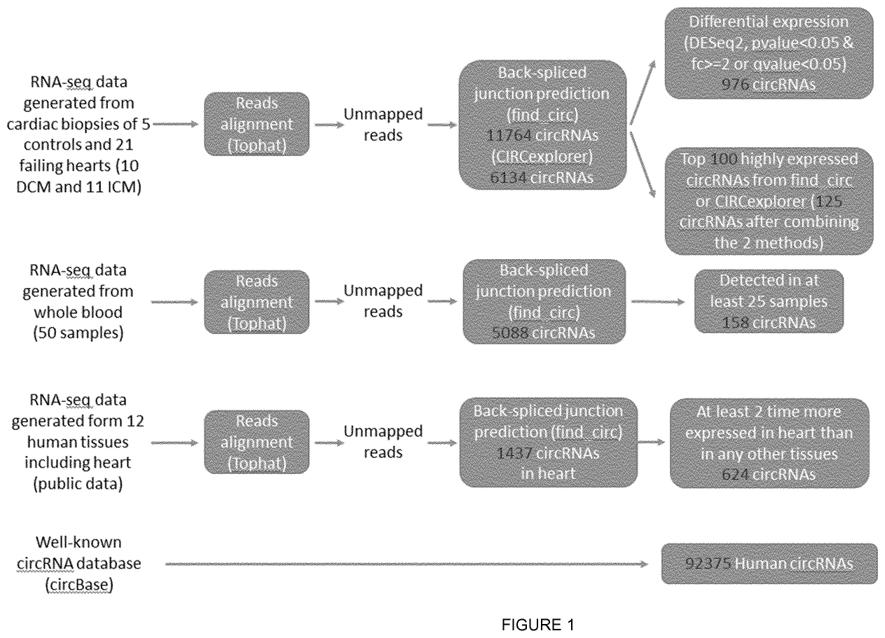

More particularly, such methods for identifying novel biomarkers for the prognosis and/or diagnosis of heart failure may comprise identifying circRNAs which are (i) differentially expressed in tissue samples from failing and non-failing hearts, (ii) highly expressed and cardiac-enriched and (iii) expressed in blood samples. Preferably, identification methods may include the steps as shown in the pipeline of FIG. 1 and as clarified in the example section below.

In the identification methods as envisaged hereinabove the expression of one or more circRNAs is determined in a sample of a subject. The subject is preferably a warm-blooded animal, more preferably a mammal, most particularly a human subject, but it can be envisaged that the methods provided herein are equally suitable for methods applied to subjects such as, e.g., non-human primates, equines, canines, felines, ovines, porcines, and the like.

In particular embodiments, the sample used to determine the biomarker is obtained from heart tissue. Most particularly the sample is a cardiac biopsy taken from the right ventricle and/or the septum. In particular embodiments, the sample size is at least 1 mm, at least 2 mm, at least 3 mm, at least 4 mm, at least 5 mm. Methods for taking cardiac biopsies are known in the art and include cardiac catheterization and/or cardiothoracic surgery. In particular embodiments, the tissue sample is snap frozen upon collection of the sample at a temperature of at least −70° C. or at least −80° C.

In particular embodiments, the sample is selected from whole blood, plasma, serum, whole blood cells, red blood cells, white blood cells (e.g., peripheral blood mononuclear cells), saliva and urine. Most particularly the sample is a cell-containing sample. In particular embodiments, the sample is a whole blood cells sample. In a further embodiment, the sample has been enriched in white blood cells.

Methods for extracting non-coding RNA, including circular RNA from tissues samples, including cardiac tissue, are known in the art and include steps of homogenizing the tissue by, for example, grinding, shearing, beating, shocking or combinations thereof.

After the tissue is homogenised, the remaining cells which are still intact may be lysed by, for example, mechanical homogenization, liquid homogenization, sonication, freeze-thaw cycles or manual grinding. These homogenization and/or lysing steps may be performed in a liquid buffer solution. After cell lysis, non-coding RNA can be extracted using commercially available kits and according to the manufacturer's instructions.

The above aspects and embodiments are further supported by the following non-limiting examples.

### EXAMPLES

### Example 1: circRNAs as Biomarkers and Therapeutic Targets of Cardiovascular Diseases

### Materials & Methods

### Human Cardiac Biopsies

Cardiac biopsies were obtained from 21 explanted failing hearts and 5 non-failing control hearts. Among failing hearts, 10 had a dilated cardiomyopathy (DCM) and 11 had an ischemic cardiomyopathy (ICM). Donors of non-failing hearts had either a head injury (n=2) or a subarachnoid haemorrhage (n=3). The protocol has been approved by the Local Ethics Committee at Cardinal Stefan Wyszynski Institute of Cardiology under the approval number IK-NP-0021-48/846/13 (Apr. 9, 2013). Neither donors nor their relatives completed National Refusal List. Biopsies were obtained from the left ventricle, the right ventricle and the septum, were snap frozen separately, and were stored at −80° C. until RNA extraction and sequencing.

### Whole Blood Samples

Whole blood samples were obtained from 50 patients after resuscitation from out-of-hospital cardiac arrest. The protocol has been approved by the national research ethics board (National Committee for Ethics in Research) and informed consent has been obtained from all subjects or their legal representatives. Blood samples for determination of circRNA expression levels were harvested in PAXgene™ tubes 48 hours after cardiac arrest. Tubes were stored at −20° C. until RNA extraction and sequencing.

### RNA Isolation

The snap frozen heart samples were homogenized in Lysis Binding Buffer (mirVana isolation kit, Life technologies) for extraction of total RNA using the mirVana isolation kit (Life technologies, Merelbeke, Belgium) according to manufacturer's instructions. On-column DNase I digestion (Qiagen, Venlo, The Netherlands) was performed to eliminate potential contamination with genomic DNA. Concentration and integrity of RNA were assessed using a Nanodrop spectrophotometer (Nanodrop products, Wilmington, USA) and a 2100 Bioanalyzer (Agilent technologies, Santa Clara, USA), respectively. Total RNA was extracted from whole blood samples which were collected in PAXgene™ tubes with the PAXgene™ blood RNA kit (Qiagen, Venlo, Netherlands) as described by the manufacturer. Extracted RNA was further purified and concentrated using the RNeasy® MinElute™ kit (Qiagen). To extract total RNA from subtypes of leukocytes, cells were lysed in TriReagent® (Sigma, Bornem, Belgium) and RNA was extracted using the RNeasy® Micro kit (Qiagen).

### RNA Sequencing

Total RNAs extracted from 26 cardiac biopsies and 50 whole blood samples were sequenced using the IIlumina™ platform.

### Pipeline of Novel circRNA Prediction (FIG. 1)

All RNA-seq data of 26 cardiac biopsies were aligned to human reference genome (hg19) using Tophat 2.1.0 as described in Trapnell C. et al. (Trapnell C. et al. Differential gene and transcript expression analysis of RNA-seq experiments with TopHat and Cufflinks. Nature protocols 2012; 7:562-78). The resulting unmapped reads were subjected to back-spliced junction prediction and circRNAs were identified using prediction algorithms. More particularly, 11764 sequences were annotated as circRNAs using find_circ and 6134 sequences were annotated as circRNAs using CIRCExplorer, as previously described in Hansen T. B. et al. (Hansen T. B. et al. Comparison of circular RNA prediction tools. Nucl. Acids Res. 2016; 44 (6): e58.). From these circRNAs (11764+6134), 976 circRNAs were differentially expressed (DESeq2, pvalue<0.05 and fc≥2 or qvalue<0.05) (referred to as differentially expressed or “DE”). Furthermore, the combination of the 100 highest expressed circRNAs obtained by using prediction algorithm find_circ and the 100 highest expressed circRNAs obtained by using prediction algorithm CIRCExplorer, lead to a total of 125 circRNAs (referred to as “high”) (FIG. 1).

Similarly, RNA-seq reads generated from 50 whole blood samples and RNA-seq reads generated from 12 different human tissues including heart tissue (obtained from public data) were aligned to human reference genome (hg19), the obtained unmapped reads were subjected to back-spliced junction prediction and circRNAs were predicted by find_circ. This approach led to the identification of 5088 circRNAs in the whole blood samples, of which 158 circRNAs were detected in at least 25 samples (referred to as “blood”), and to the identification of 1437 circRNAs in heart tissue of which 624 circRNAs were at least 2 times more expressed in heart than in any other tissue (referred to as “heart”) (FIG. 1).

The circRNAs obtained from these three different sources ((i) cardiac biopsies obtained by the Applicants, (ii) whole blood samples obtained by the Applicants and (iii) publically available data on 12 different human tissues including heart) and the circRNAs from the circBase, which is a well-known circRNA database comprising 92375 human circRNAs, were subjected to further selection processes to select the most interesting circRNAs as biomarker and/or therapeutic target of cardiovascular diseases.

### Results

### Selection of circRNAs as Biomarkers for Heart Failure

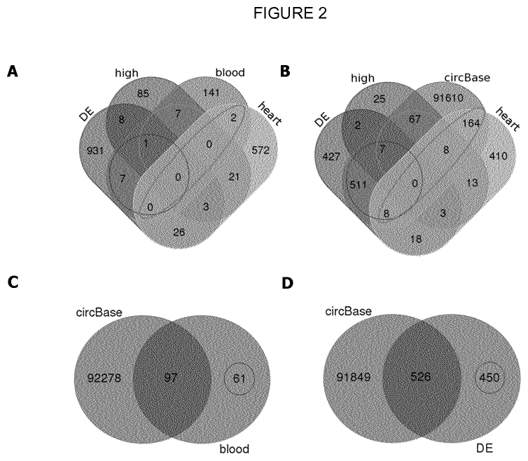

Selection 1 was performed based on circRNAs that were (i) expressed in blood cells, (thereby being especially interesting as biomarker), (ii) differentially expressed (DE) between failing and non-failing human hearts or between ICM (ischemic cardiomyopathy) and DCM (dilated cardiomyopathy), and/or (iii) highly expressed or enriched in the heart. More particularly, 8 circRNAs were DE in HF patients or between ICM and DCM (DE) and were detected in blood samples. Additionally, 8 circRNAs were found to be highly expressed in heart (“high”) and were detected in blood samples (“blood”). This selection criterium was included because not all DE circRNAs can be detected by RNA-seq and highly expressed circRNAs in heart have more chance to be good biomarkers of heart diseases. Furthermore, 2 circRNAs were found to be enriched in heart (“heart”) and were detected in blood (“blood”). This selection criterium was included because some circRNAs can be lowly expressed in general but are still relatively highly expressed in heart. circRNAs fulfilling this selection criterium also have more chance to be good biomarkers of heart diseases. Overall, selection 1 led to the identification of a total of 17 circRNAs that can act as biomarkers for heart failure in a patient as described herein (FIG. 2A; Table 1, FIG. 3).

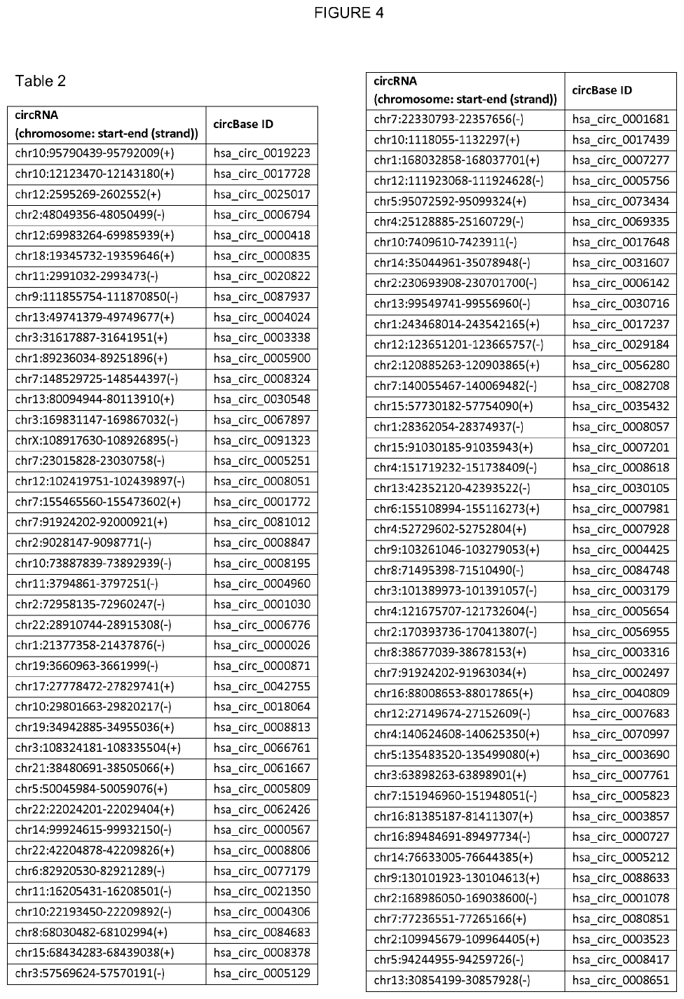

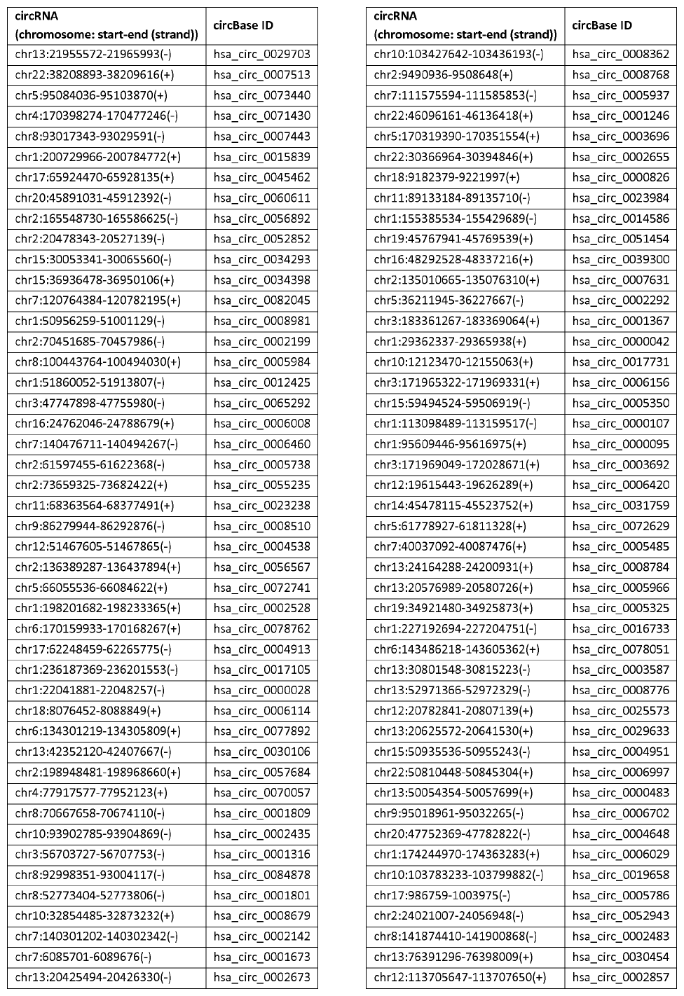

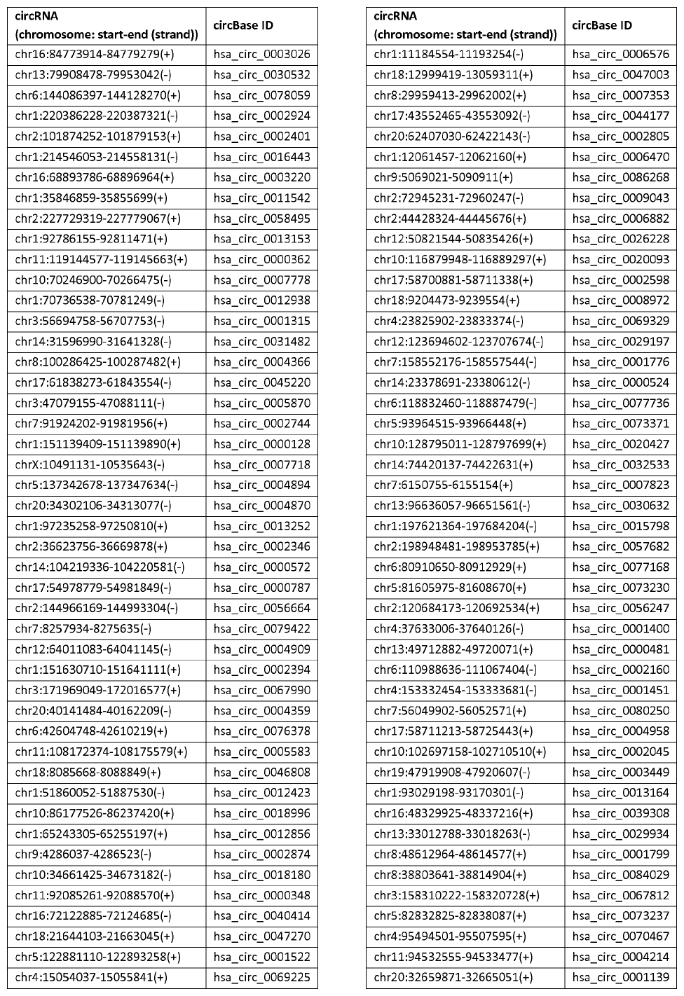

Since not all blood circRNAs might be detectable in the blood samples used in this experiment, a second selection was based on circRNAs of the circBase database, more particularly, the circRNAs identified from tissues or cells different from heart tissue. In view thereof, selection 2 was performed based on circRNAs that were (i) known in circBase, (ii) DE between failing and non-failing human hearts or between ICM and DCM and/or (iii) highly expressed or enriched in the heart. More particularly, 526 circRNAs were DE in HF patients or between ICM and DCM (“DE”) and found in circBase (“circBase”). Furthermore, 82 circRNAs were highly expressed in heart (“high”) and found in circBase (“circBase”). This selection criteria was included because not all DE circRNAs can be detected by RNA-seq and circRNAs, which are highly expressed in heart, have more chance to be good biomarkers of heart diseases. Additionally, 180 circRNAs were found to be enriched in heart (“heart”) and were found in circBase (“circBase”). This selection criteria was included because some circRNAs could be lowly expressed in general but are still relatively highly expressed in heart. This type of circRNAs also have more chance to be good biomarkers of heart diseases. Overall, selection 2 to led to the identification of 765 circRNAs that can act serve as biomarkers for heart failure in a patient as described herein (FIG. 2B; Table 2, FIG. 4).

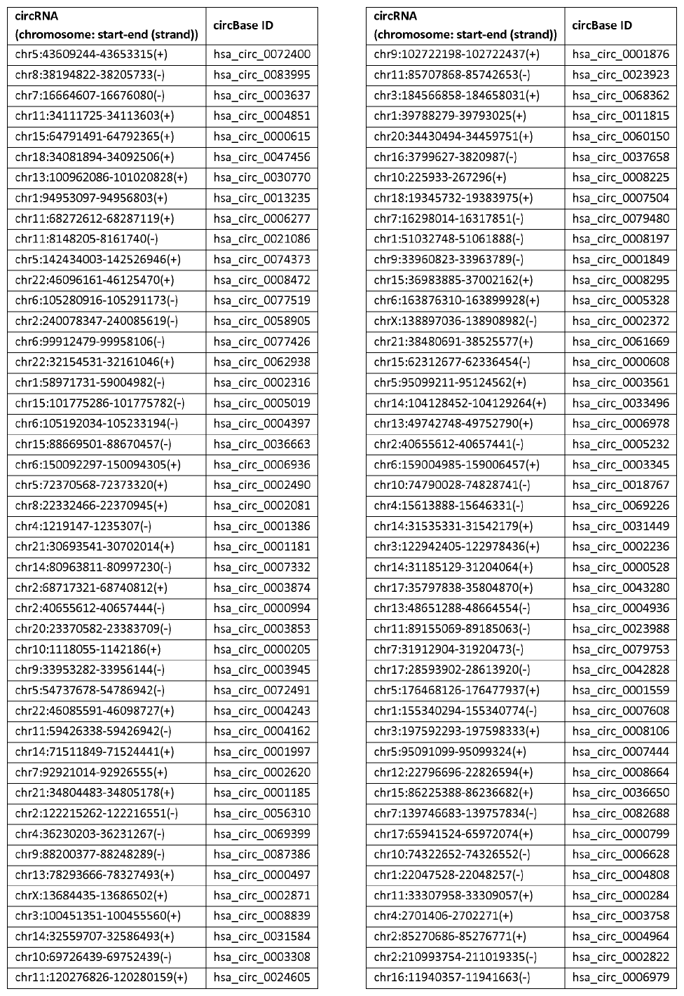

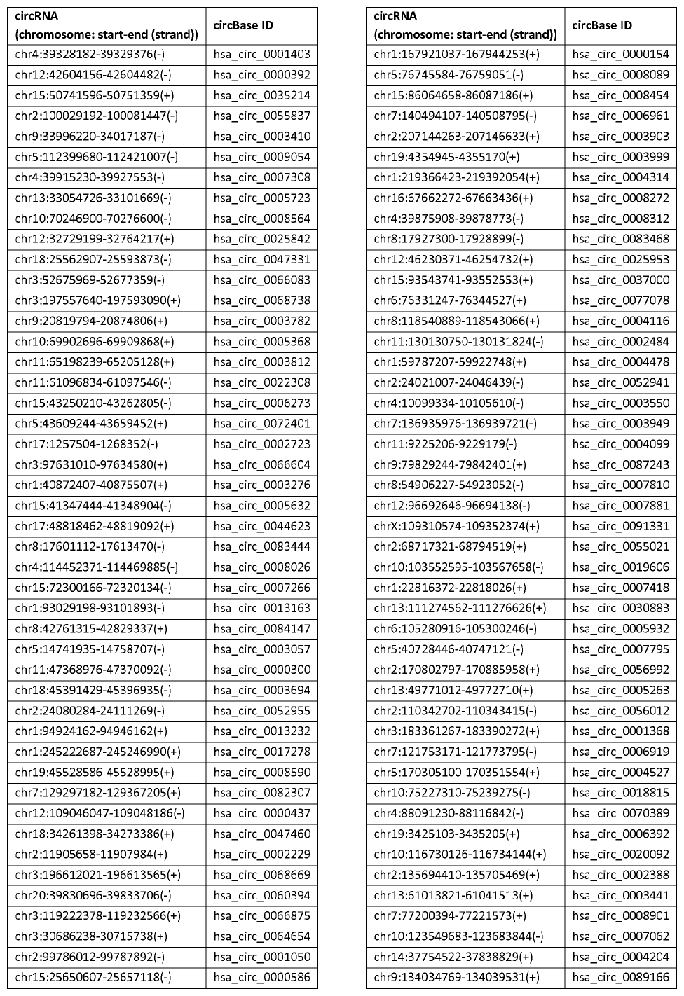

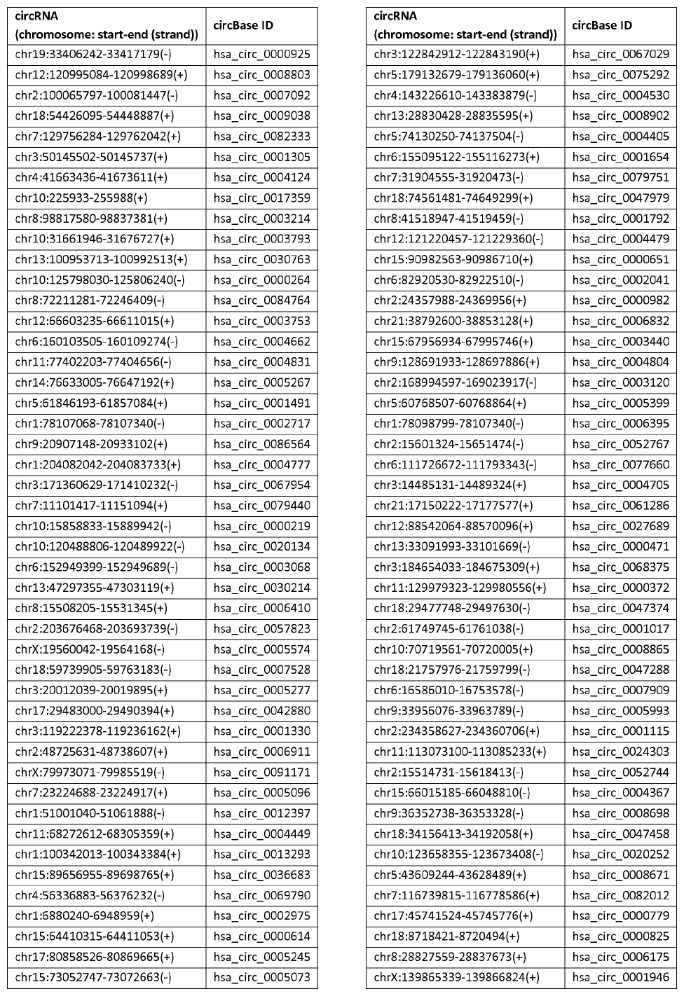

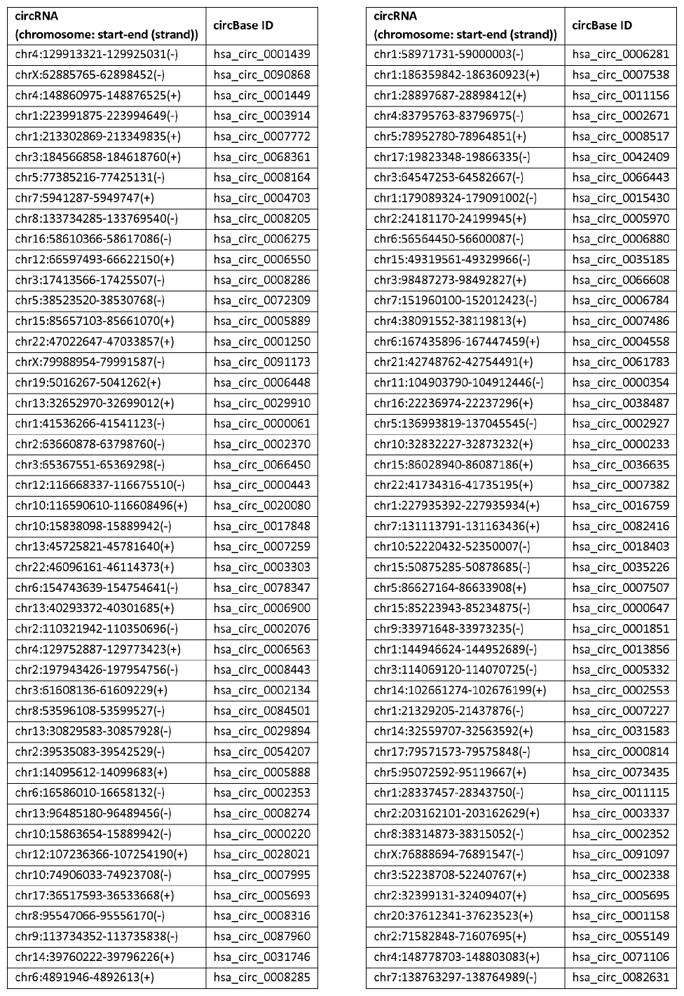

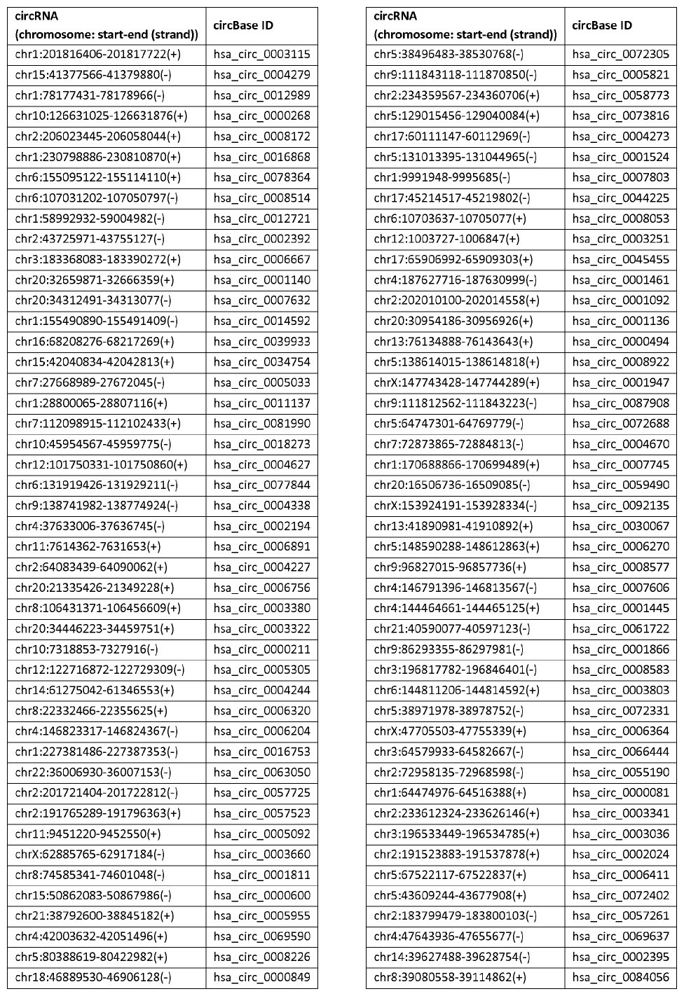

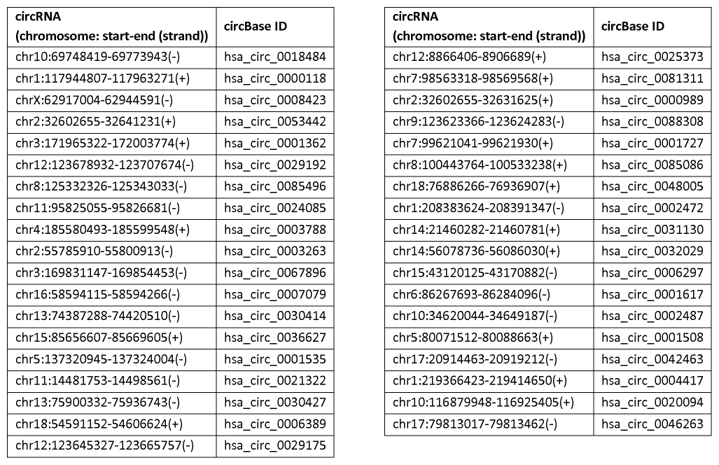

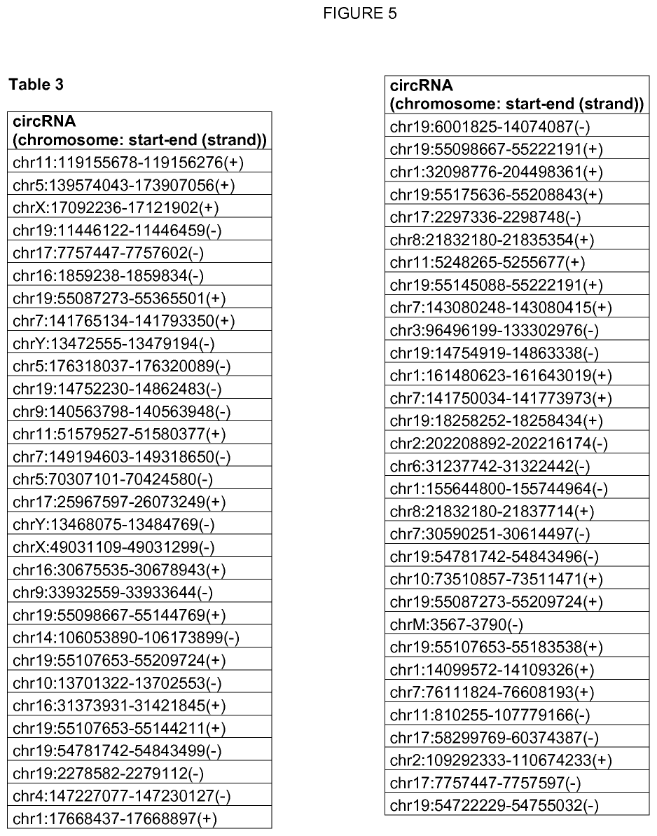

Selection 3 was performed based on circRNAs that were expressed in blood cells, but which were not known in circBase. 61 completely novel circRNAs were identified, which can act as biomarkers for heart failure in a patient as discribed herein (FIG. 2C; Table 3, FIG. 5).

Selection 4 was performed based on circRNAs that were DE between failing and non-failing human hearts or between ICM and DCM but which were not known in circBase.

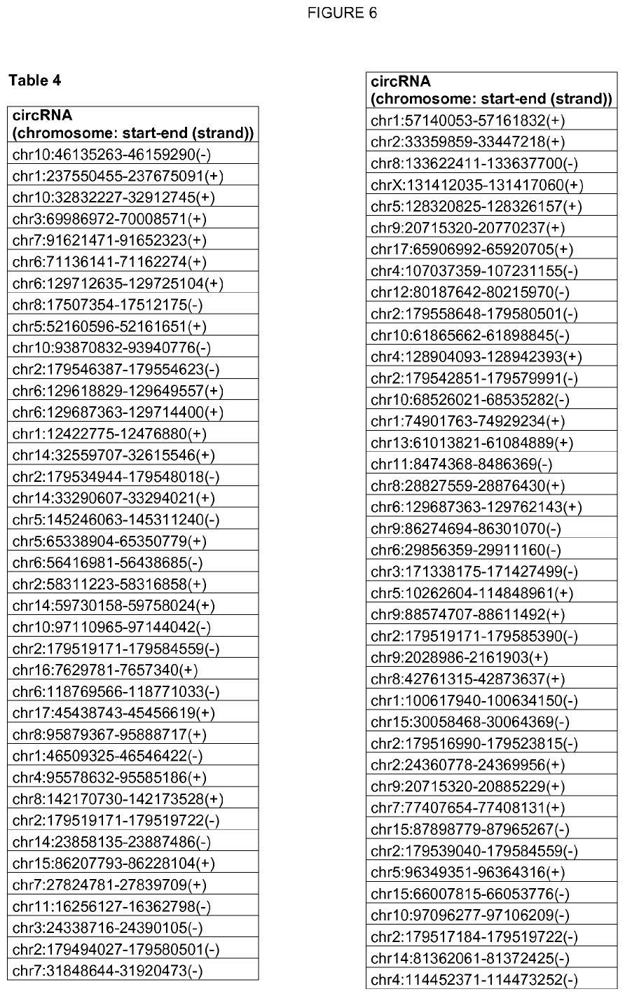

This led to the selection of 450 completely novel circRNAs which can act as biomarkers for heart failure in a patient as described herein and which are particularly interesting as novel therapeutic targets of heart failure (FIG. 2D; Table 4, FIG. 6).

### CircRNAs are Differentially Expressed in the Failing Heart

It was verified that the circRNAs of Table 1 are expressed in the heart and it was tested whether the expression thereof is regulated during heart failure (ICM or DCM).

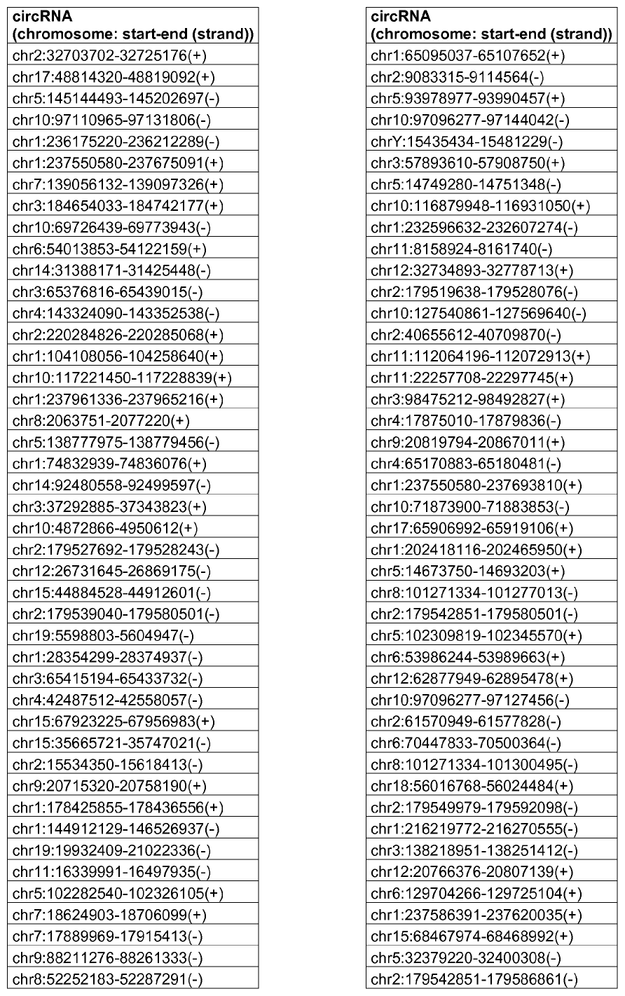

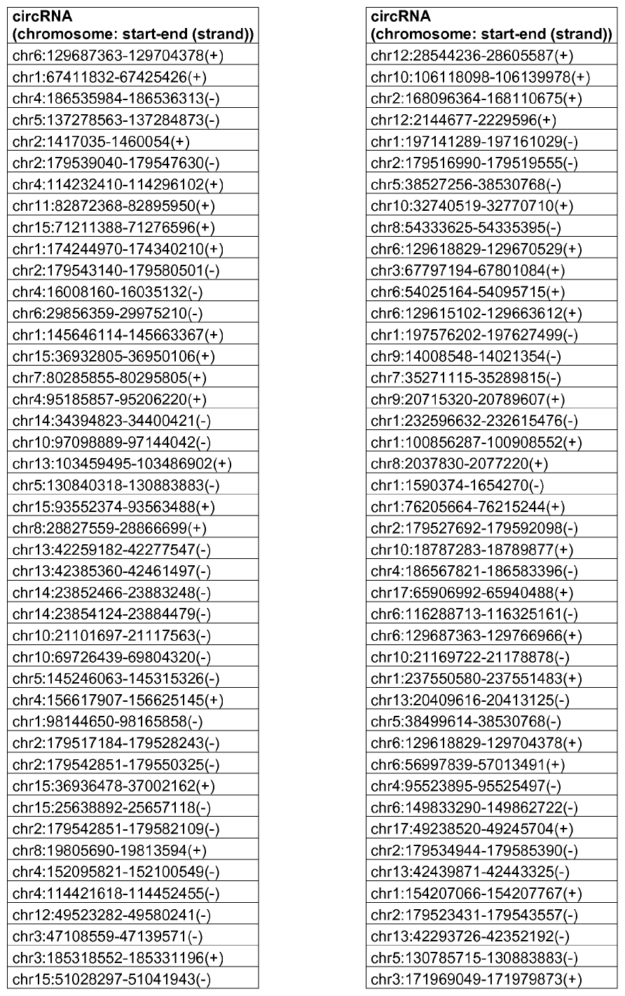

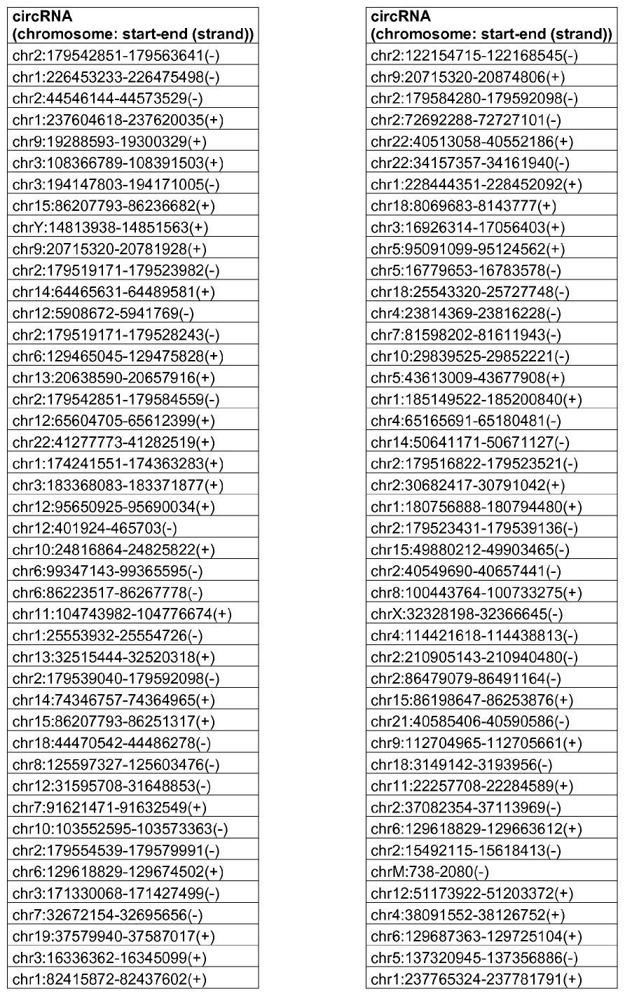

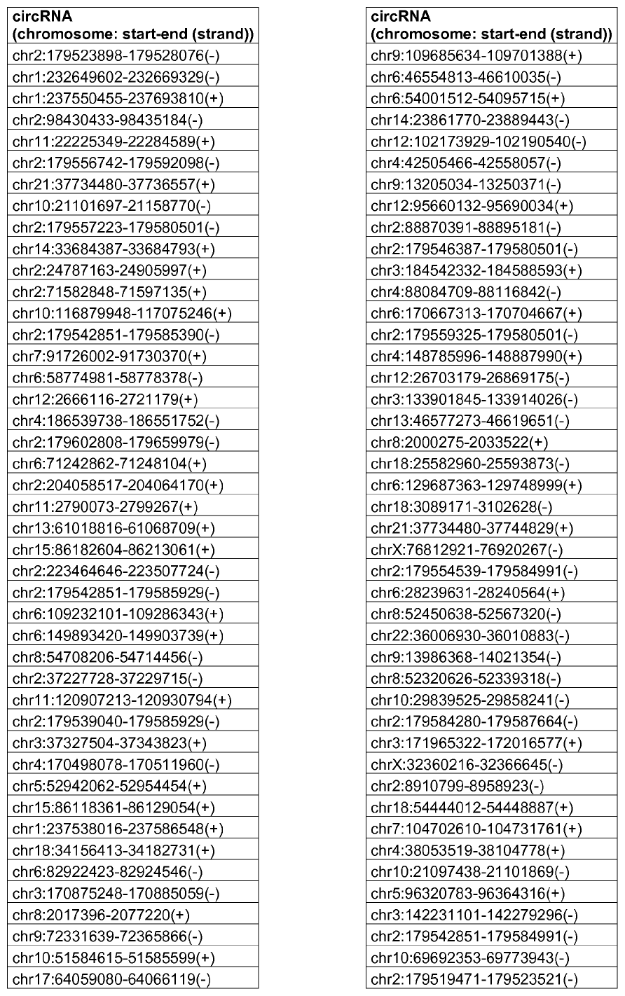

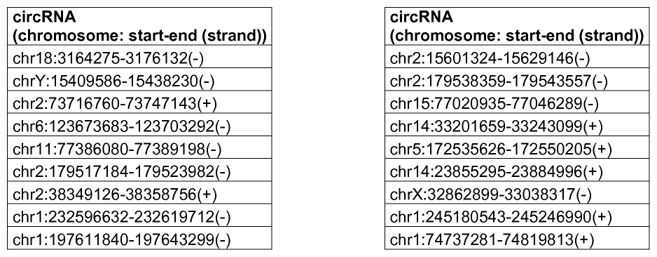

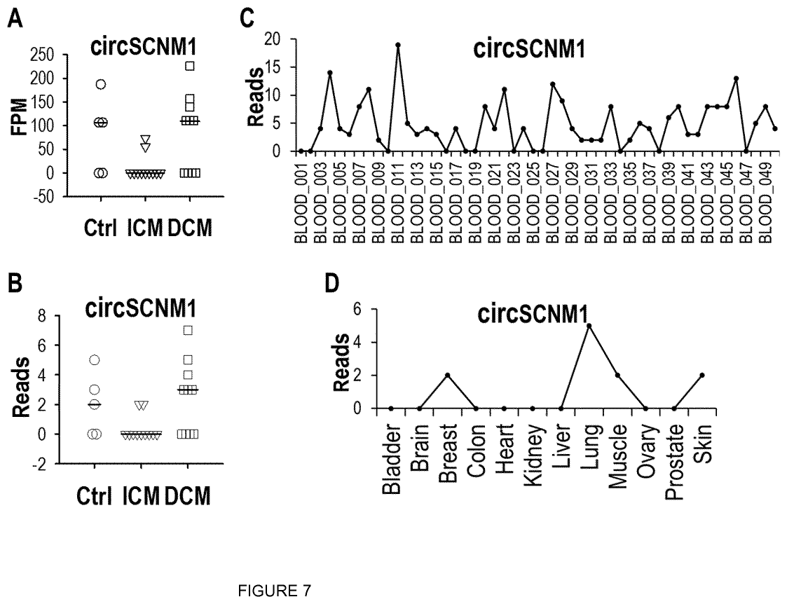

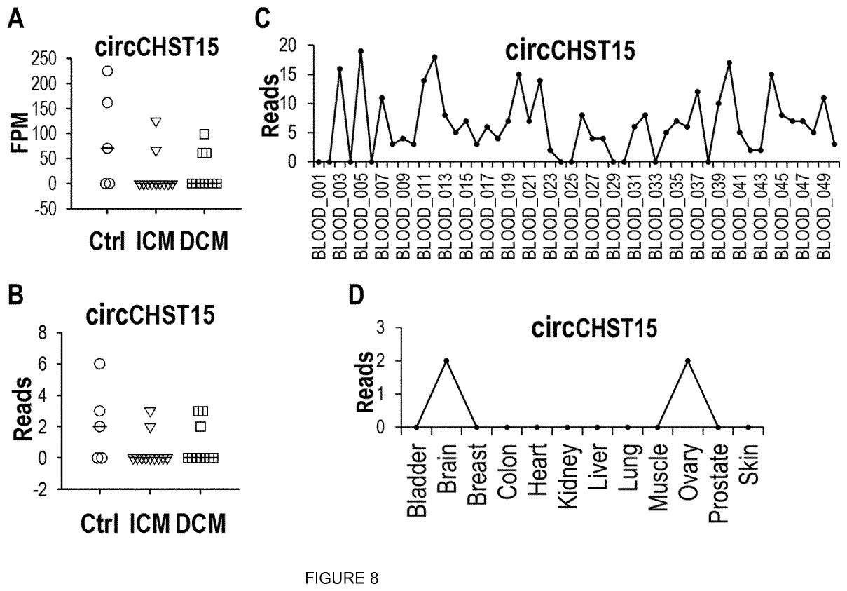

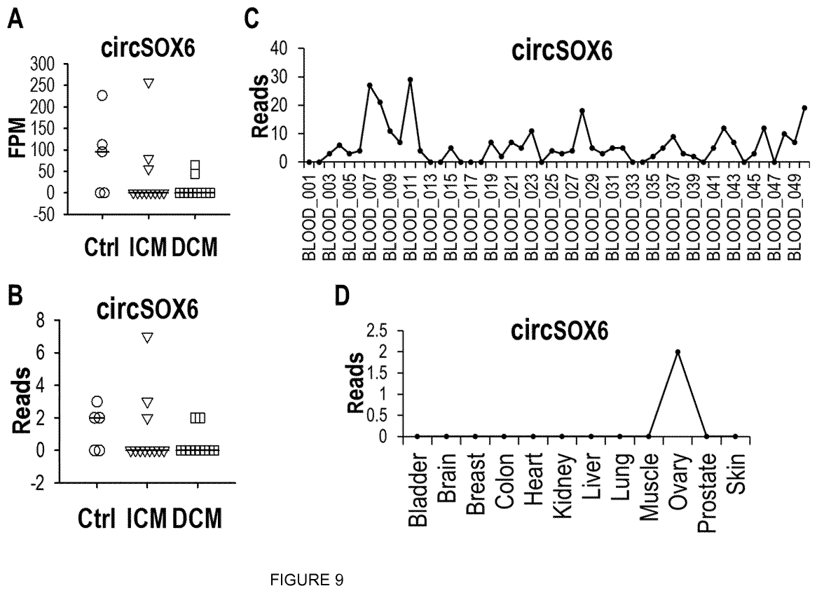

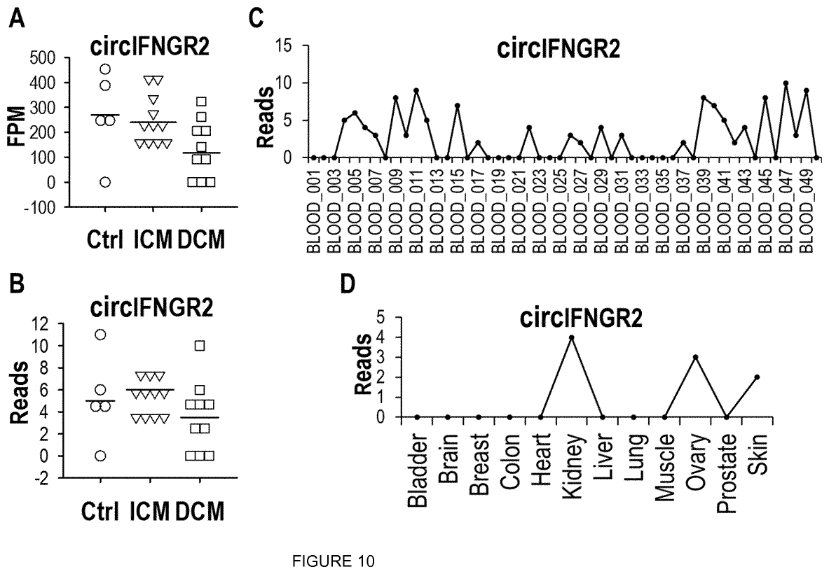

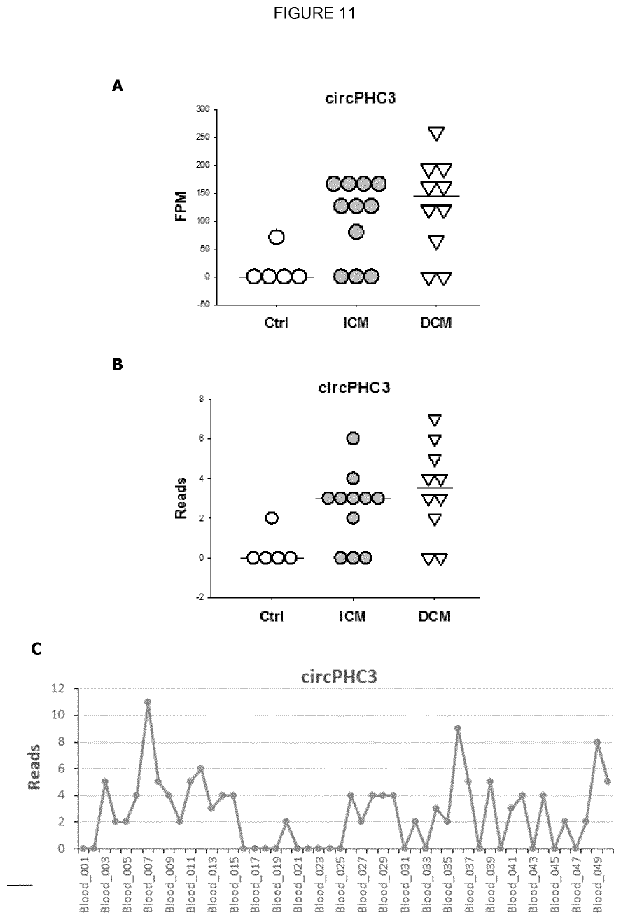

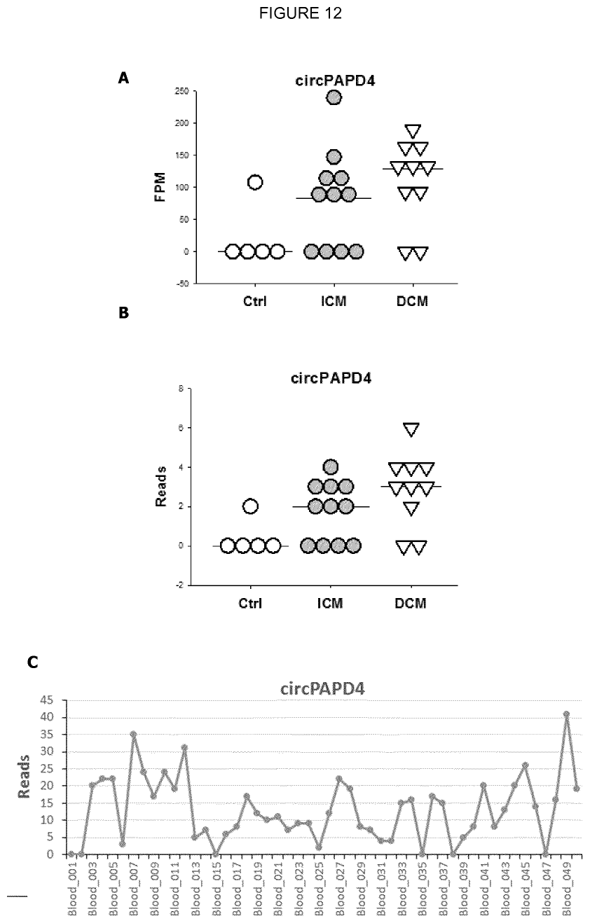

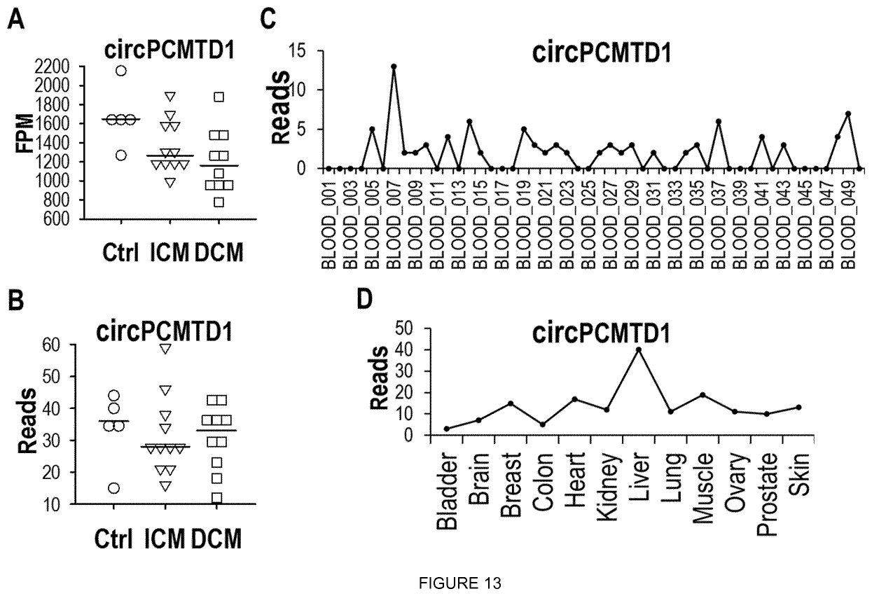

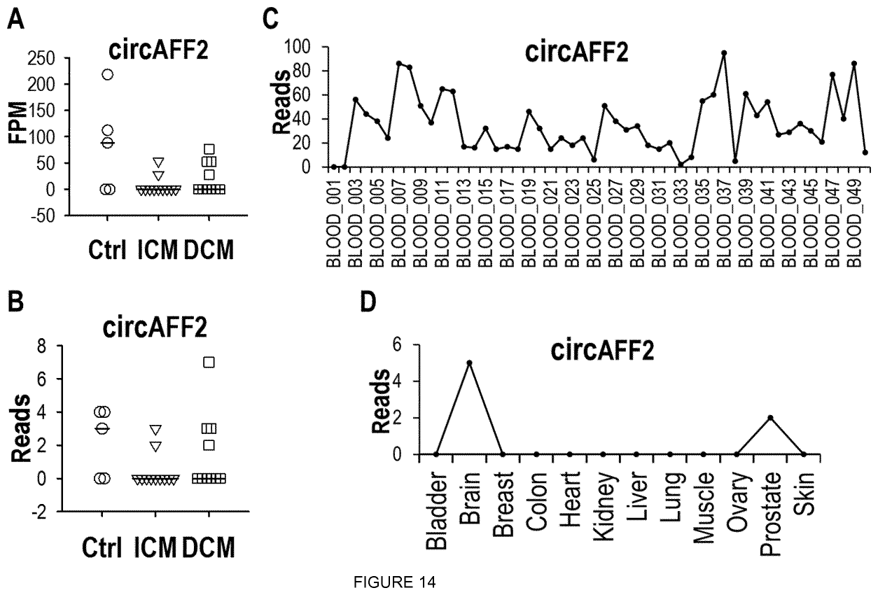

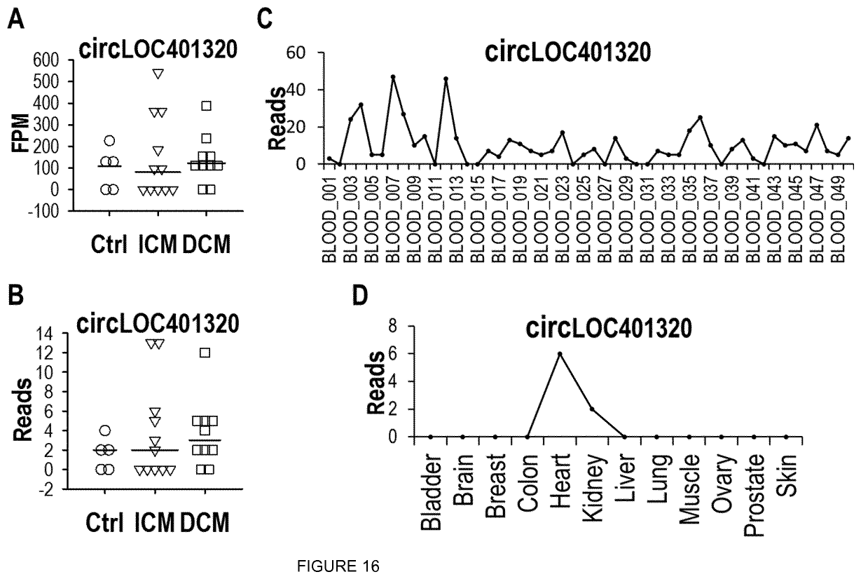

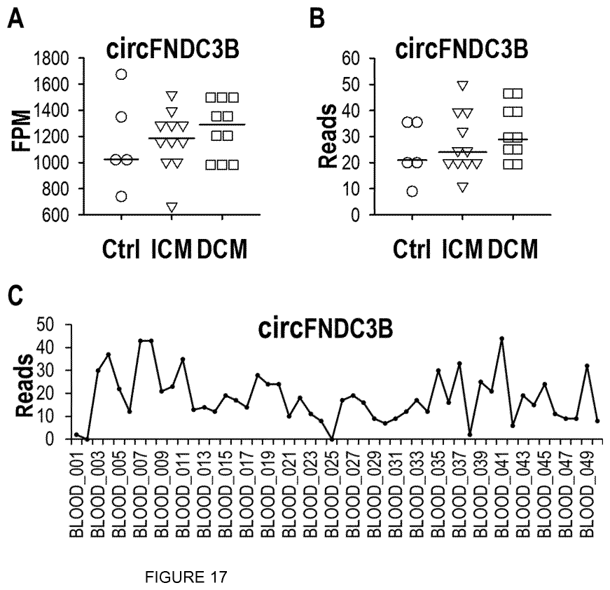

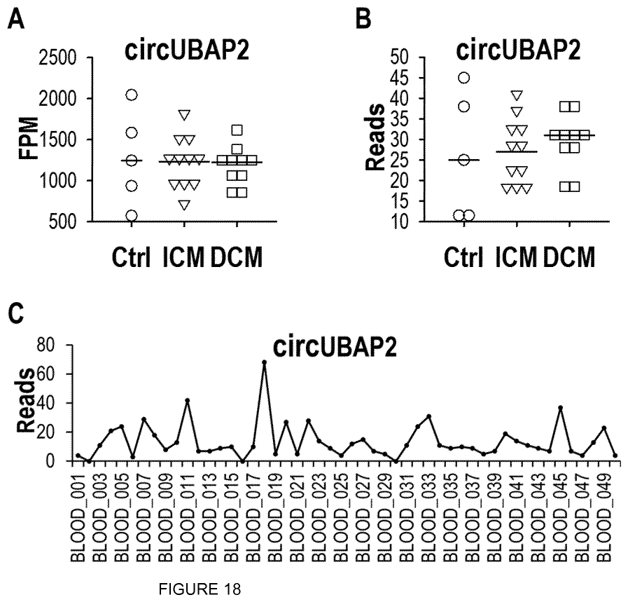

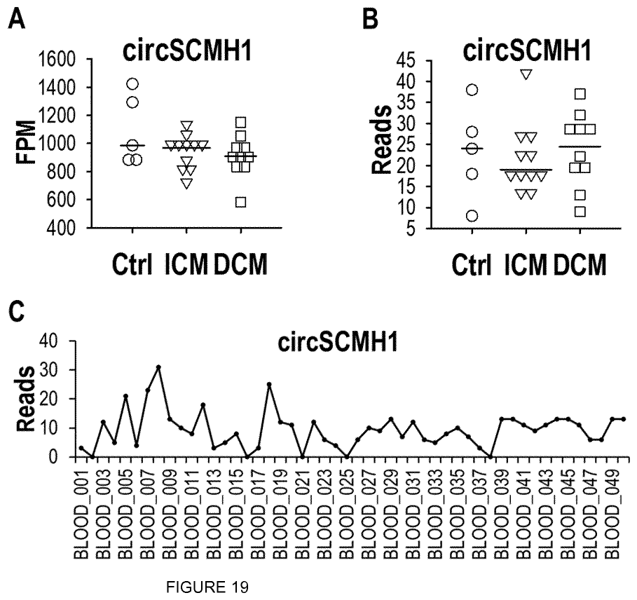

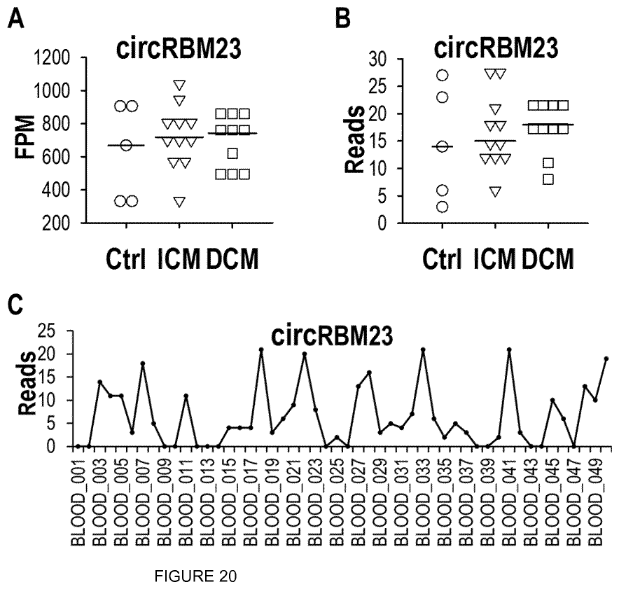

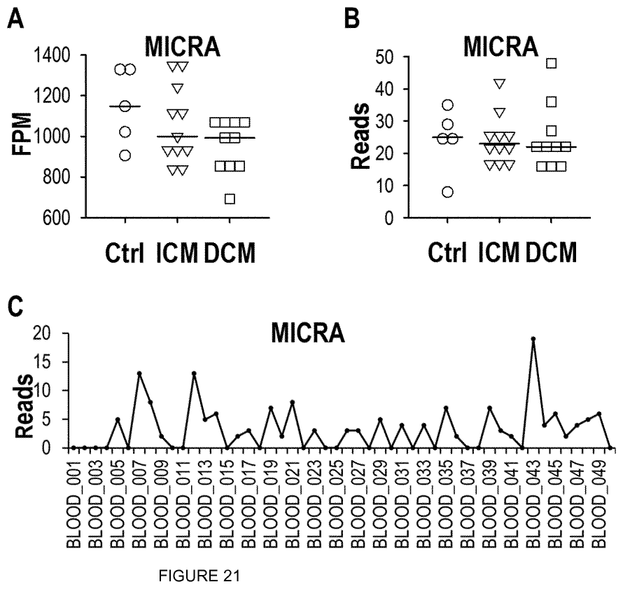

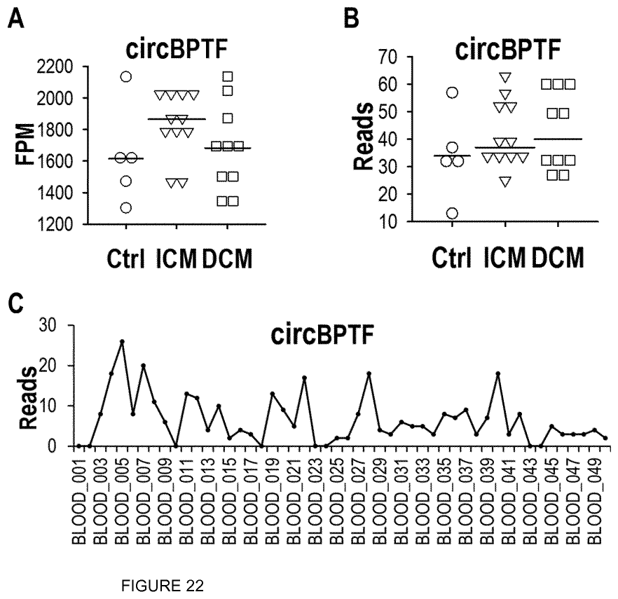

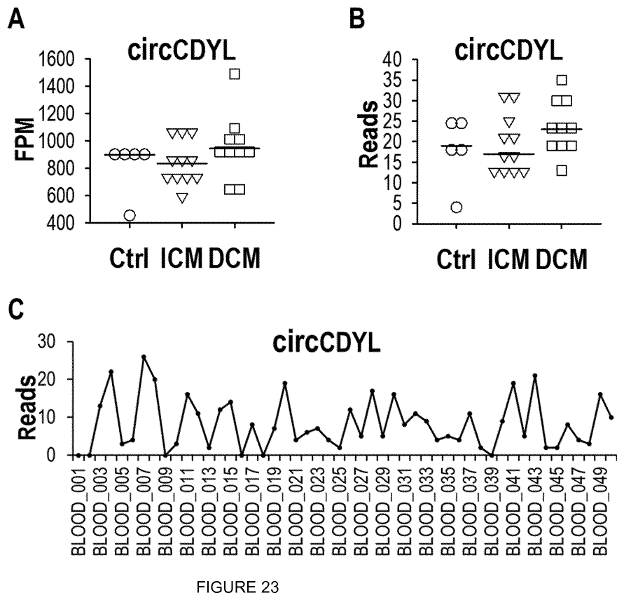

In general, circSCNM1 (FIG. 7A-B), circCHST15 (FIG. 8A-B), circSOX6 (FIG. 9A-B), circIFNGR2 (FIG. 10A-B), circPHC3 (FIG. 11A-B), circPAPD4 (FIG. 12A-B), circAFF2 (FIG. 14A-B) were only lowly expressed in heart tissue; circCASP1/CARD16 was not detected in heart tissue (data not shown) and circPCMTD1 (FIG. 13A-B), circLOC401320 (FIG. 16A-B), circFNDC3B (FIG. 17 A-B), circUBAP2 (FIG. 18 A-B), circSCMH1 (FIG. 19 A-B), circRBM23 (FIG. 20 A-B), MICRA (FIG. 21 A-B), circBPTF (FIG. 22 A-B) and circCDYL (FIG. 23 A-B) were well expressed in heart tissue.

Normalised circSCNM1 expression levels were decreased in ICM patients, but not in DCM patients, when compared to control (FIG. 7 A-B). Normalised circCHST15 (FIG. 8 A-B), circSOX6 (FIG. 9 A-B), circPCMTD1 (FIG. 13 A-B), circAFF2 (FIG. 14 A-B) and MICRA (FIG. 21 A-B) expression levels were decreased in both ICM and DCM patients when compared to control. Normalised circIFNGR2 (FIG. 10 A-B) and circSCMH1 (FIG. 19 A-B) expression levels were decreased in DCM patients when compared to control and ICM patients. Normalised circPHC3 (FIG. 11 A-B), circPAPD4 (FIG. 12 A-B), circFNDC3B (FIG. 17 A-B), circRBM23 (FIG. 20 A-B) and circBPTF (FIG. 22 A-B) expression levels were increased in ICM and DCM patients when compared to control. Normalised circLOC401320 (FIG. 16 A-B) and circUBAP2 (FIG. 18 A-B) expression levels were unaltered in ICM and DCM patients compared to control.

Normalised circCDYL expression levels were decreased in ICM patients, but increased in DCM patients when compared to control (FIG. 23 A-B).

These data showing that the 17 circRNAs have distinct features (association with heart failure, high expression/enrichment in the heart, expression in blood) illustrate that all 17 circRNAs of Table 1 can provide complementary information in a multivariable prediction model. Thus, all 17 circRNAs are biomarkers that can be used in the diagnosis of heart failure or prediction of decompensation.

### Expression of circRNAs in Blood Samples

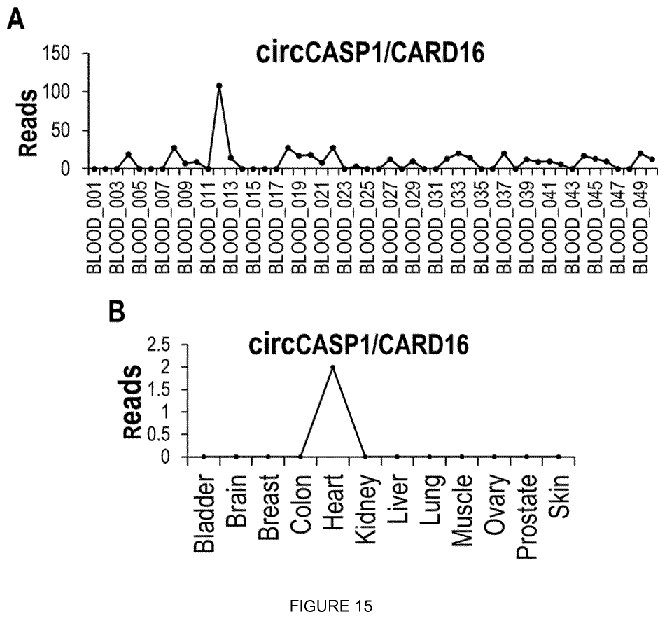

FIGS. 7C, 8C, 9C, 100, 11C, 12C, 13A, 14C, 15C, 16C, 17C, 18C, 19C, 20C, 21C, 22C and 23C illustrate the expression of circSCNM1, circCHST15, circSOX6, circIFNGR2, circPHC3, circPAPD4, circPCMTD1, circAFF2, circCASP1/CARD16, circLOC401320, circFNDC3B, circUBAP2, circSCMH1, circRBM23, MICRA, circBPTF and circCDYL respectively, in 50 blood samples as assessed by RNA-seq (expressed in raw reads). circSCNM1, circCHST15, circSOX6, circPHC3, circPAPD4, circAFF2, circLOC401320, circFNDC3B, circUBAP2, circSCMH1, circRBM23, circBPTF and circCDYL were detected in most of the blood samples, while circIFNGR2, circPCMTD1, circCASP1/CARD16 and MICRA were detected in half of the blood samples.

These data show that the identified circRNAs of Table 1 which can act as biomarkers for heart failure can also be detected in blood and hence, only a non-invasive and convenient blood sample from a patient would be required for making a prognosis, diagnosis and/or determining a method of treatment.

### Expression of circRNAs in Different Organs

FIGS. 7D, 8D, 9D, 10D, 13B, 14D, 15D and 16D illustrate the expression of circSCNM1, circCHST15, circSOX6, circIFNGR2, circPCMTD1, circAFF2, circCASP1/CARD16 and circLOC401320 respectively, in 12 different human tissues.

These data were obtained from a public dataset (expressed in raw reads).

In human tissues, circSCNM1 was expressed in breast, lung, muscle and skin, particularly in lung (FIG. 7D), circCHST15 was mostly expressed in brain and ovary (FIG. 8D), circSOX6 was mostly expressed in ovary (FIG. 9D), circIFNGR2 was expressed in kidney, ovary and skin, particularly in kidney (FIG. 10D), circPCMTD1 was expressed in all 12 human tissues, particularly in liver, circCASP1/CARD16 was mainly expressed in heart and circLOC401320 was detected in heart and kidney, particularly in heart. The absence of circSCNM1, circCHST15, circSOX6 and circIFNGR2 in heart could be explained by examining the expression of these circRNAs only one sample of each tissue.

These data showing that circCASP1/CARD16 and circLOC401320 are preferentially expressed in the heart illustrate that one or both of these circRNAs may reflect heart function and may provide useful information in a multivariable prediction model. However, this does not exclude that other circRNAs also provide information on heart function.

### Example 2: Further Selection of circRNAs as Biomarkers and Therapeutic Targets of Cardiovascular Diseases and Validation Thereof

### Materials & Methods

### Human Cardiac Biopsies

Cardiac biopsies were obtained from 43 explanted failing hearts and 23 non-failing control hearts. Among failing hearts, 26 had a dilated cardiomyopathy (DCM) and 17 had an ischemic cardiomyopathy (ICM). Donors of non-failing hearts had either a head injury (n=8) or a subarachnoid haemorrhage (n=15). The protocol has been approved by the Local Ethics Committee at Cardinal Stefan Wyszynski Institute of Cardiology under the approval number IK-NP-0021-48/846/13 (Apr. 9, 2013). Neither donors nor their relatives completed National Refusal List. Biopsies were obtained from the left ventricle, the right ventricle and the septum, were snap frozen separately, and were stored at −80° C. until RNA extraction and sequencing.

### RNA Isolation

As described in example 1

### Quantitative PCR

One microgram of total RNA was reverse-transcribed using the Superscript II RT kit (Life technologies). Real-time quantitative PCR was performed in a CFX96 apparatus (Biorad) with IQ SYBR Green Supermix (Biorad) and divergent primers designed with the Beacon Designer software (Premier Biosoft). Glyceraldehyde-3-phosphate deshydrogenase (GAPDH) was chosen as a housekeeping gene for normalization. Expression levels were calculated by the relative quantification method (ΔΔCt) using the CFX Manager 2.1 (Bio-Rad).

### RNase R Treatment

Total RNA from the left ventricle of 3 control and 3 failing hearts were treated with RNase R (Epicentre) or mock and RNAseOut (Invitrogen) according to manufacturer's instructions. Treated RNA was reverse-transcribed using Superscript II (Invitrogen).

Quantitative PCR was performed as described above. Relative resistance of circRNAs to RNAse degradation was calculated as the ratio of circRNA resistance and linear Sf3a1 resistance: 2^{(Mock C_t of circRNA-RNase R C_t of circRNA)}/2^{(Mock C_t of Sf3a1-RNase R C_t of Sf3a1)}. The higher the ratio, the more resistant to RNase R. PCR amplification products were purified using MinElute PCR Purification Kit (Qiagen) and sequenced using BigDye Terminator v1.1 Cycle Sequencing Kit (Thermo Fisher Scientific).

### Results

### Selection of circRNAs as Biomarkers for Heart Failure

circRNAs were selected as biomarkers for heart failure by selections 1-4 (referred to as “selection groups 1-4” in Table 5 (FIG. 32)) as described in Example 1.

### Selection of circRNAs Validation in Left Ventricular (LV) biopsies Using Quantitative PCR (qPCR)

The following three selection criteria were applied to select circRNAs for validation in LV biopsies: (i) similar expression profiles between the RNA-seq data of the inventors and public datasets, (ii) high expression level, and (iii) number of circRNAs to be validated kept to a reasonable number. This was achieved by:
(i) selecting circRNAs with a similar expression profiles in public RNA-seq data generated from LV of 2 control subjects (non diseased), 2 subjects with HCM (hypertrophic cardiomyopathy), and 2 subjects with DCM (dilated cardiomyopathy) (Khan et al. (2016). RBM20 Regulates Circular RNA Production From the Titin Gene. Circulation research. 119, 996-1003), as well as from LV of 3 control hearts, 1 DCM heart, 1 ICM (ischemic cardiomyopathy) heart and 1 HCM (hypertrophic cardiomyopathy) heart (Tan et al. (2017) A landscape of circular RNA expression in the human heart. Cardiovasc Res 113, 298-309) and the RNA-seq data obtained as described in Example 1;
(ii) selecting circRNAs with a relatively high expression level in the RNA-seq data obtained as described in Example 1 (median counts>10); and
(iii) a reasonable number (<20) of circRNAs to measure.

Using these selection criteria, 15 circRNAs (as listed Table 5 (FIG. 32)) were selected for validation.

### Confirmation of the Circular Form of the 15 Selected Candidate circRNAs

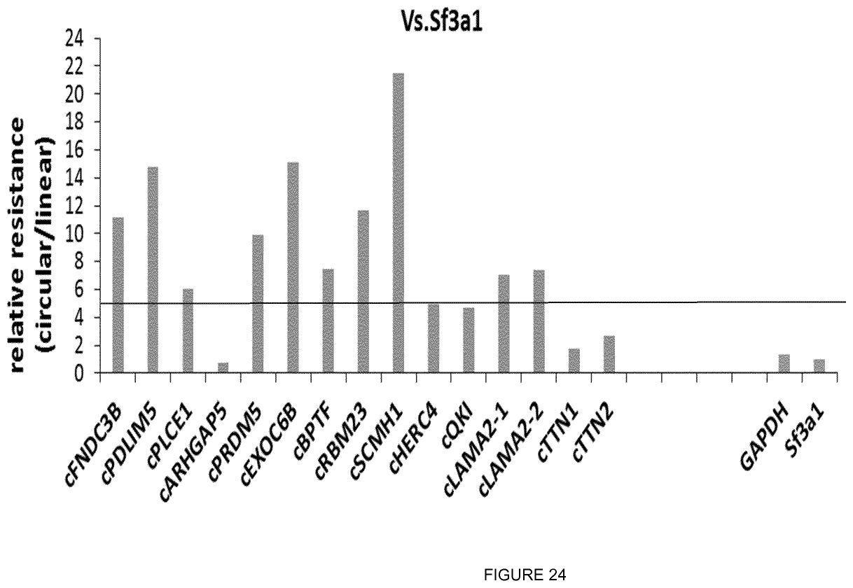

An RNase R resistance assay was performed to evaluate if the 15 circRNAs selected for validation were (at least partly) under a circular form. RNase R is an exoribonuclease that digests linear RNAs, but not circRNAs. The RNase R resistance, which reflects the proportion of circular forms over linear forms, was determined using a relative resistance of 5 as a threshold value. RNA with relative resistance above this threshold was considered resistant to RNAse R, and hence circular. Subsequently, the circRNAs resistant to RNAse R were measured using qPCR. 10/15 circRNAs were found to be resistant to RNase R (FIG. 24).

### Confirmation of Back Splice Junction

Sanger sequencing of PCR products was performed to check whether selected circRNAs contain a back splice site, which is a typical feature of circRNAs.

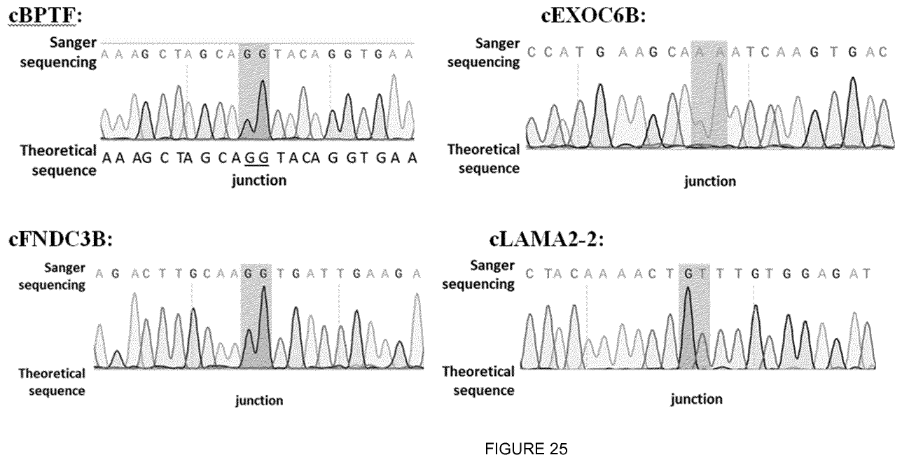

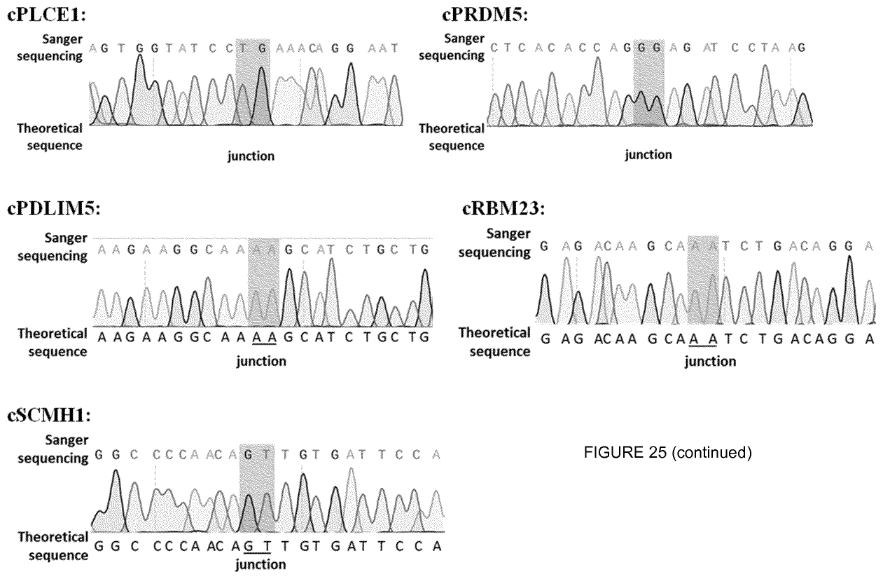

Back-splice junctions of 9 RNAse R-resistant circRNAs were confirmed by Sanger sequencing (FIG. 25).

### Differential Expression of circRNAs in All Cardiac Biopsies

To validate the association between selected circRNAs and heart disease observed in the human cardiac biopsies from Example 1, qPCR for the 9 RNAse R-resistant circRNAs, which show back-splice junctions, were performed for a total of 66 LV biopsies (23 controls, 26 DCM and 17 ICM, referred to herein as “large cohort study”). 6/9 circRNAs were differentially expressed in the 66 LV biopsies.

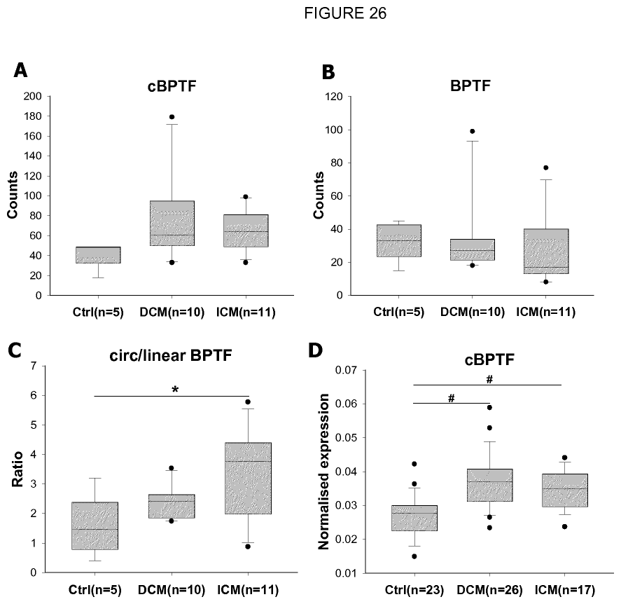

FIGS. 26A, 27A, 28A, 29A, 30A, and 31A represent the expression of circRNA, FIGS. 26B, 27B, 28B, 29B, 30B, and 31B represent the expression of the circRNA's host linear gene and FIGS. 26C, 27C, 28C, 29C, 30C, and 31C represent the ratio of the expression of circRNA and its host linear gene, in 5 control (ctrl), 11 ICM and 10 DCM samples as assessed by RNA-seq. FIGS. 26D, 27D, 28D, 29D, 30D, and 31D represent the expression of circRNA in control (ctrl), ICM and DCM as assessed by qPCR in 66 LV biopsies (23 controls, 26 DCM and 17 ICM).

The expression of circular BPTF was higher in human cardiac biopsies compared to its host linear gene (FIG. 26A-B). Furthermore, the ratio of circular over linear BPTF was increased in HF (i.e., in DCM or ICM samples, especially in ICM) (FIG. 26C). The increase of circBPTF in both DCM and ICM compared to controls was validated by qPCR in the large cohort study (FIG. 26D).

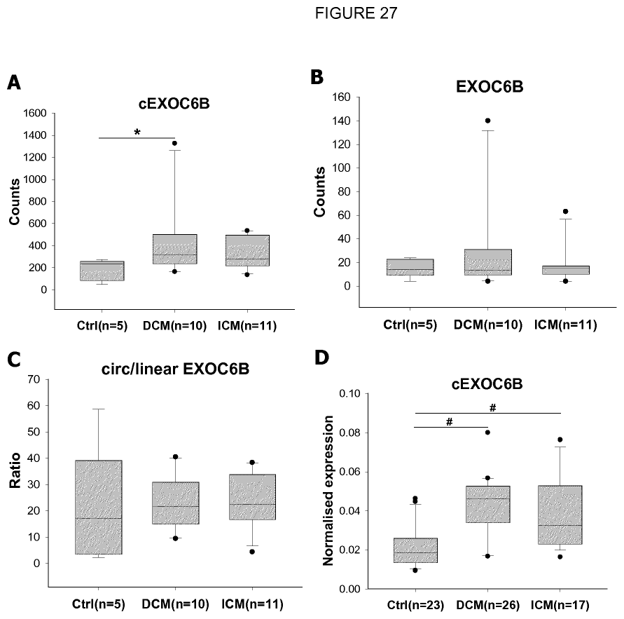

The expression of circular EXOC6B in all human cardiac biopsies was higher than its host linear gene (FIG. 27A-B). The ratio of circular over linear EXOC6B was slightly increased in HF (i.e., in DCM or ICM samples) (FIG. 27C). The increase of circEXOC6B in both DCM and ICM compared to controls was validated by qPCR in the large cohort study (FIG. 27D).

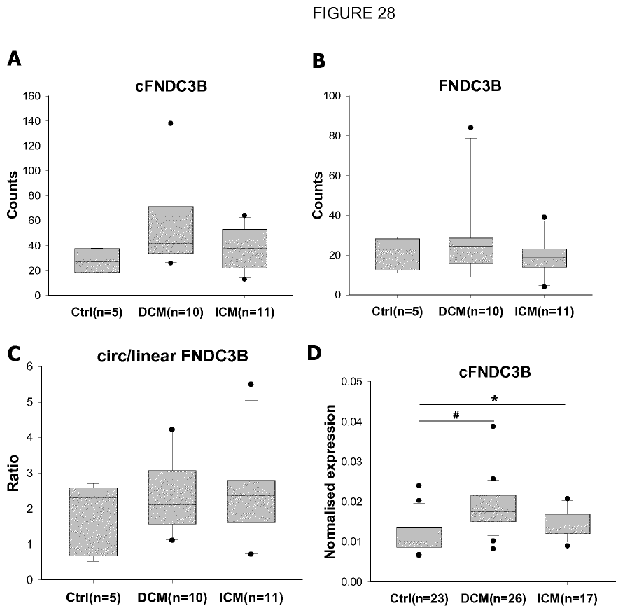

The expression of circular FNDC3B was slightly higher than its host linear gene in all human cardiac biopsies (FIG. 28A-B). The ratio of circular over linear FNDC3B was not increased in HF (i.e., in DCM or ICM samples) (FIG. 28C). The increase of circFNDC3B in DCM compared to controls was validated by qPCR in the large cohort study (FIG. 28D).

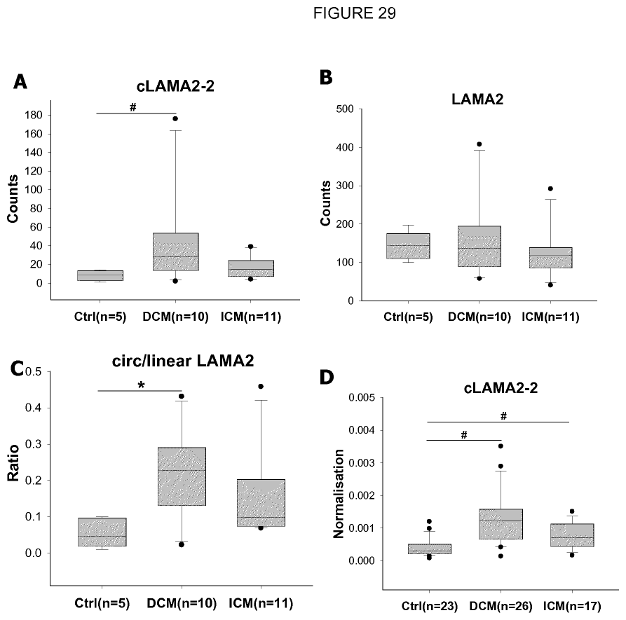

The expression of circular LAMA2-2 was lower than its host linear gene in all human cardiac biopsies (FIG. 29A-B). The ratio of circular LAMA2-2 and linear LAMA2 was increased in DCM and ICM samples, especially in DCM samples (FIG. 29C). The increase of circLAMA2-2 expression in both DCM and ICM compared to controls was validated by qPCR in the large cohort study (FIG. 29D).

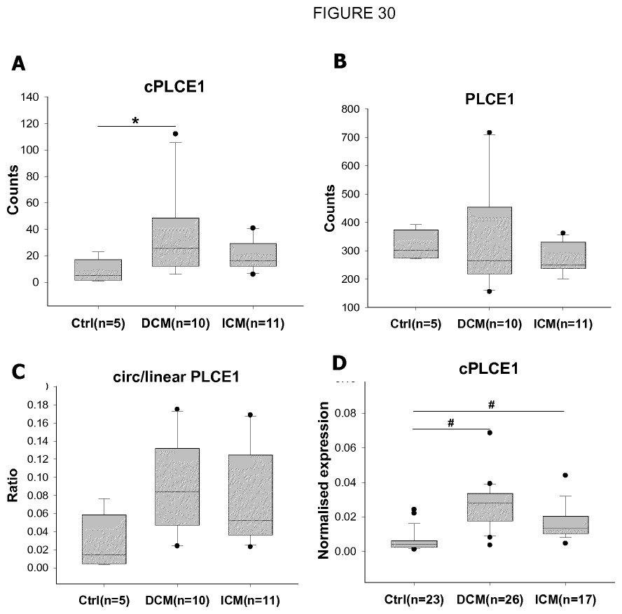

The expression of circular PLCE1 was lower than its host linear gene gene in all human cardiac biopsies (FIG. 30A-B).The ratio of circular and linear PLCE1 was increased in HF (FIG. 30C). The increase of circPLCE1 expression in both DCM and ICM samples compared to controls was validated by qPCR in the large cohort study (FIG. 30D).

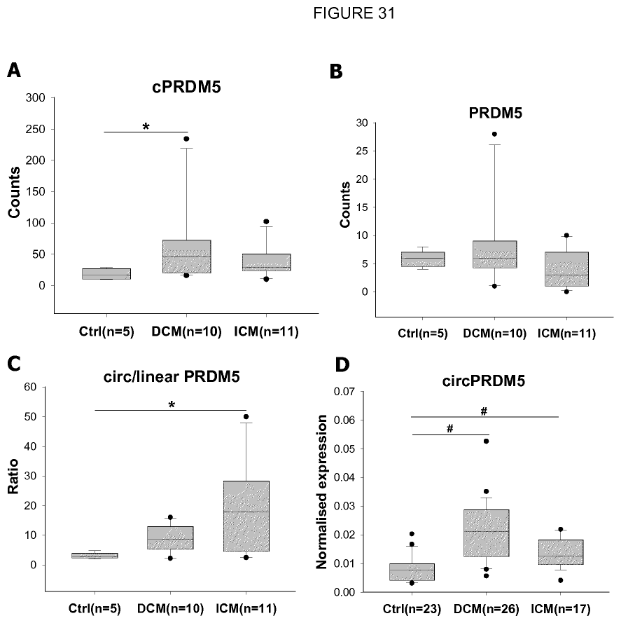

The expression of circular PRDM5 was higher than its host linear gene in all human cardiac biopsies (FIG. 31A-B). The ratio of circular and linear PRDM5 was increased in HF (FIG. 31C). The increase of circPRDM5 in both DCM and ICM samples compared to controls was validated by qPCR in the large cohort study (FIG. 31D).

### CONCLUSION

6 circRNAs, namely cBPTF, cEXOC6B, cFNDC3B, cLAMA2-2, cPLCE1 and cPRDM5, were identified with reproducible associations with HF, and are therefore key biomarkers for the diagnosis of HF and/or predicting the clinical evolution of HF in a patient.

All of these 6 circRNAs were resistant to RNase R and were significantly up-regulated in both DCM and IDM samples compared to control samples in the large cohort study of 66 LV biopsies. Furthermore, their back-splice junctions were confirmed by Sanger sequencing. Additionally, the expression of cBPTF, cEXOC6B and cPRDM5 was significantly higher than their linear gene in human cardiac biopsies. For BPTF, LAMA2 and PRDM5, the ratio of the circular form and their linear gene was also increased in ICM or DCM in RNA-seq data generated from 26 LV biopsies. cLAMA2-2 is not registered in circBase, version of May, 2017 (in which it was indicated that the most recent update of the database took place in December 2015) and therefore a novel circRNA.

1-20. (canceled)

21. A method for diagnosing heart failure and/or predicting the clinical evolution of heart failure in a patient in vitro or ex vivo comprising determining the expression of one or more circular RNAs (circRNAs) selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654) and diagnosing heart failure and/or predicting the clinical evolution based thereon.

22. The method according to claim 21, wherein said one or more circRNAs is cFNDC3B and one or more circRNAs selected from cBPTF, cEXOC6B, cLAMA2-2, cPLCE1 and cPRDM5.

23. The method according to claim 21, further comprising determining the expression of one or more circRNAs selected from Table 1, Table 2, Table 3, or Table 4, in addition to the one or more circRNAs selected from cFNDC3B, cBPTF, cEXOC6B, cLAMA2-2, cPLCE1 and cPRDM5.

24. The method according to claim 21, which comprises (i) determining the expression level of said one or more of circRNAs in a sample of said patient; and optionally (ii) comparing said expression level to the expression level of said one or more circRNAs in a control sample, wherein said diagnosing of heart failure and/or predicting of the clinical evolution of heart failure in said patient is based on the differential expression of said one or more circRNAs.

25. The method according to claim 21, wherein said expression level is determined by RT-PCR assay, a sequencing-based assay, quantitative nuclease-protection assay (qNPA) or a microarray assay.

26. The method according to claim 21, wherein the diagnosis further comprises assessing one or more clinical factors in said patient and combining said assessment of said one or more clinical factors and the expression of said one or more circRNAs in said prediction or diagnosis, wherein said clinical factor is selected from the group consisting of breathlessness, exertional dyspnea, orthopnea, paroxysmal nocturnal dyspnea, dyspnea at rest, acute pulmonary edema, chest pain/pressure and palpitations or non-cardiac symptoms such as anorexia, nausea, weight loss, bloating, fatigue, weakness, oliguria, nocturia, cerebral symptoms of varying severity, ranging from anxiety to memory impairment and confusion, fluid retention, cardiac rhythm disturbances, prolonged corrected QT interval and complete Left Bundle Branch Block.

27. The method according to claim 21, which further comprises assessing one or more other biomarkers in said patient and combining said assessment of said one or more other biomarkers and the expression of said one or more circRNAs in said prediction or diagnosis, wherein, wherein said one or more other biomarkers is selected from the group consisting of long non-coding RNAs, microRNAs, CPK, cTnT, Nt-pro-BNP, MMP9, VEGFB, THBS1, and P1GF.

28. The method according to claim 21, which comprises determining expression of all six of cFNDC3B, cBPTF, cEXOC6B, cLAMA2-2, cPLCE1 and cPRDM5.

29. The method according to claim 21, wherein said sample is a whole blood sample.

30. A method for the treatment or prevention of heart failure in a patient comprising determining, in a sample of said patient, the expression of one or more circular RNAs (circRNAs) selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654), and, upon increased or decreased expression of one or more of said circular RNAs, treating said patient with an ACE-inhibitor.

31. A system for diagnosing heart failure and/or predicting the clinical evolution of heart failure in a patient, the system comprising: a storage memory for storing data associated with a sample obtained from the patient, wherein the data comprises quantitative expression data for one or more circRNAs (circRNAs) selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654) and a processor communicatively coupled to the storage memory for analyzing the dataset, configured to analyze the expression level of said one or more circRNAs and to diagnose heart failure or determine the risk of a poor outcome after heart failure based thereon.

32. A computer-readable storage medium storing computer-executable program code, which, when run on a computer allows storing of the data and the analysis of the data in the system according to claim 31.

33. A kit for diagnosing and/or predicting the outcome of heart failure in a patient, comprising reagents for determining quantitative expression of one or more circRNAs (circRNAs) selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654) in a sample of a patient and instructions for using said reagents for determining said quantitative expression.

34. A method for selecting an optimal treatment for a patient with heart failure said method comprising determining the risk a poor clinical evolution of said patient with heart failure by determining the expression in a sample of said patient of one or more circRNAs (circRNAs) selected from cFNDC3B (hsa_circ_0006156), cBPTF (hsa_circ_0000799), cEXOC6B (hsa_circ_0009043) cLAMA2-2, cPLCE1 (hsa_circ_0019223) and cPRDM5 (hsa_circ_0005654) and selecting the treatment for said patient based thereon.

35. A method for the treatment or prevention of heart failure in a patient, the method comprising, administering to said patient, an agent capable of inhibiting expression or activity of one or more circular RNAs (circRNAs) selected from cFNDC3B, cBPTF, cEXOC6B, cLAMA2-2, cPLCE1 and cPRDM5.

36. The method of claim 35, which comprises, prior to said treatment, determining in a sample of said patient, increased or decreased expression of said one or more of said circular RNAs (circRNAs).

37. The method of claim 36, which further comprises assessing one or more clinical factors in said patient and combining said assessment of said one or more clinical factors and the expression of said one or more circRNAs.

38. The method of claim 35, which further comprises determining in a sample of said patient the expression of one or more circRNAs selected from Table 1, Table 2, Table 3, or Table 4, in addition to the one or more circRNAs selected from cFNDC3B, cBPTF, cEXOC6B, cLAMA2-2, cPLCE1 and cPRDM5.
